(19) United States
(12) Patent Application Publication
(10) Pub. No.: US 2024/0269199 A1
(43) Pub. Date: Aug. 15, 2024

(54) COMPOSITIONS FOR CONTROL OF HUMAN AND ANIMAL PARASITIC NEMATODES AND METHODS OF USE

(71) Applicant: UNIVERSITY OF MASSACHUSETTS, Boston, MA (US)

(72) Inventors: Raffi Van Aroian, Shrewsbury, MA (US); Kelly Flanagan, Boston, MA (US)

(21) Appl. No.: 18/415,118

(22) Filed: Jan. 17, 2024

Related U.S. Application Data
(60) Provisional application No. 63/439,759, filed on Jan. 18, 2023.

(51) Int. Cl. A61K 35/742; A61K 9/50; A61K 35/00; A61K 38/16; A61P 33/10; C12N 1/36

(52) U.S. Cl. CPC A61K 35/742; A61K 9/5089; A61K 38/164; A61P 33/10; C12N 1/36; A61K 2035/115

(57) ABSTRACT

Compositions and methods for treating or reducing the severity or likelihood of occurrence of a parasitic worm infection in a humans and domestic animals are described. The compositions include inactivated recombinant bacteria expressing a Cry14Ab crystal protein in the cytosol of the bacterium. The composition may be provided in a form suitable for oral administration. Methods include administering a therapeutically effective amount of the composition to a human or domestic animal suffering from a parasitic worm infection. The composition may also be used as feed, feed supplement, or a waste treatment.

### RELATED APPLICATIONS

The present invention claims the benefit of U.S. Provisional Application No. 63/439,759, filed Jan. 18, 2023, the contents of which are incorporated herein by reference in its entirety for all purposes.

### STATEMENT OF FEDERALLY SPONSORED RESEARCH

This invention was made with government support under Grant No. AI056189 awarded by the National Institutes of Health, and Grant No. 2016-67015-24861 awarded by the U.S. Department of Agriculture. The government has certain rights in the invention.

### BACKGROUND

Soil-transmitted helminths (nSTHs) are a group of parasitic worms infecting both humans and animals living in resource-limited settings. They are the most common infectious agents of humans in developing countries and produce a global burden of disease that exceeds better-known conditions, including malaria and tuberculosis.

STHs parasitize the GI tract of humans infect 2.3 billion of the poorest peoples and >400,000,000 of the poorest children worldwide. (Hall, A., et al. Matern Child Nutr 4 Suppl 1, 118-236 (2008)) Infected children can exhibit growth stunting, retarded cognitive development, lethargy, malnutrition, increased school absenteeism, and vulnerability to secondary infections. (Bethony, J. et al. Lancet 367, 1521-32 (2006); Hotez, P. J. Forgotten people, Forgotten diseases. (2008)) Pregnant women who are infected are at increased risk for low birth-weight babies and for maternal and infant mortality. (Brooker et al., PLoS Negl Trop Dis 2, e291 (2008)). Infected individuals have lower energy, lower productivity, and immune defects that result in increased virulence of HIV/AIDS and a higher likelihood of contracting malaria and tuberculosis (Stothard et al., Ann Trop Med Parasitol 103, 357-60 (2009); Moran, M. et al., G-finder Report (2009)); STHs thus trap large populations of the developing world in poverty. The common link of STH transmission is poor sanitation, which requires a massive investment in infrastructure and public health.

Parasitic worms also infect animals including livestock and companion animals like cows, sheep, goats, horses, pigs, poultry, dogs, and cats. Parasitic worm infections can lead to devastating economical losses to animal based farm production. Farm animals can suffer from malnutrition, tissue damage, and blood loss resulting in impaired production traits and reproduction parameters. Moreover, infected animals can transmit parasites to humans. Thus, treatment of animal helminth infection is of great commercial interest.

Conventional chemotherapy approved by the World Health Organization for STH infections in humans involves treatment with benzimidazoles (e.g., albendazole, mebendazole) or nicotinic acetylcholine receptor (nAChR) agonists (pyrantel, levamisole). (Keiser and Utzinger, JAMA 299, 1937-48 (2008)). These compounds, however, lack full efficacy against most human STH parasites. Reports in humans of resistance to both classes of drugs are increasing (e.g., Tanzania, 2010 (Stothard et al., Ann Trop Med Parasitol 103, 357-60 (2009)), potentially rendering ineffective current strategies for controlling STH infections. A notable challenge in this field is that the infected populations are among the poorest in the world, and economic incentives to develop new drugs are low (˜$700,000/year is spent to develop new drugs against human STHs (Moran, M. et al. G-finder Report (2009)). The poverty of infected populations demands that STH therapeutics be safe, effective, and also inexpensive; highly stable even in the absence of a cold chain; transportable through distribution routes to infected populations; and amenable to culturally acceptable delivery systems.

Crystal (Cry) proteins made by the soil bacterium Bacillus thuringiensis (Bt) may be candidate agents that provide safe and effective treatment of STHs. Cry proteins have been in use for 60+ years as safe, natural, organic insecticides for control of crop pests, mosquitoes, and black flies. (Roh, J. Y., et al. J MICROBIOL BIOTECHNOL 17, 547-59 (2007)). They are also effective against nematodes. (Wei, J. Z. et al. PROC NATL ACAD SCI 100, 2760-5 (2003)). Cry proteins are non-toxic to vertebrates and are EPA approved for expression in transgenic food (e.g., corn, potato). (Mohamadzadeh et al. PNAS 106, 4331-6 (2009); Betz F. S., et al. REGUL ToxICOL PHARMACOL 32, 156-73 (2000)). They are stable and cheap to mass-produce. Activity of Cry proteins against nematode plant parasites and against helminths has been described, e.g., in WO2007/062064; US2010/0024075; WO2010/053517; and US2011/0263489; see also, e.g., Li, X.-Q. et al., 2008 Biol. Control 47:97-102, which describes activity of a Cry5B protein truncated at amino acid residue 698 against C. elegans and plant parasitic nematodes.

Cry proteins can be expressed at high levels and safely delivered to humans or animals using the B. thuringiensis in the inactivated bacteria with cytosolic crystals (IBaCC system).

However, known Cry proteins are not always effective against all parasitic worms and parasitic worms can develop resistance to Cry proteins over time. Thus, there exists a need to identify new Cry proteins effective against parasitic worms that infect humans and animals.

### SUMMARY

The subject disclosure provides compositions and methods for control of parasitic worms in humans and domesticated animals. The disclosure is based on the inventors' discovery that Cry 14Ab is effective for treatment of animal parasitic nematodes.

In one aspect, a composition is provided that comprises a killed or inactivated non-sporulating bacterium that is genetically engineered to express Cry14Ab in the cytosol of the bacterium.

In certain exemplary embodiments, the killed or inactivated bacterium is genetically engineered to have a genetic mutation that results in a defect in sporulation such that Cry14Ab is trapped in the cytosol of the bacterium.

In certain exemplary embodiments, the expression of the gene encoding Cry14Ab is under control of a non-sporulation-specific promoter. In certain exemplary embodiments, the promoter is a Cry3A, GerA, GNAT, or TadA promoter.

In certain exemplary embodiments, the bacterium is a Gram-positive bacterium. In certain exemplary embodiments, the bacterium is a species of Bacillus. In certain exemplary embodiments, the bacterium is Bacillus thuringiensis (Bt).

In certain exemplary embodiments, the bacterium has a genetic mutation, wherein the genetic mutation is a deletion or inactivation of one or more genes resulting in a defect of sporulation; wherein the one or more genes resulting in a defect in sporulation is selected from the group consisting of: kinA, kinB, spo0A, spo0B, spo0E, spo0F, spo0J, spo0M spoIIB, spoIID, spoIIE, spoIIF, spoIIG, spoIIL, spoJIM, spoIIIA, spoIIIB, spoIIIE, spoIVA, spoIVC, spoIVD, spoVG, spoVK, spoVL, spoVM, spoVN, spoVP, spoVQ, spoVID, σH, σF, σE, σG, and σK. In certain exemplary embodiments, the genetic mutation is a deletion or inactivation of the spo0A gene.

In certain exemplary embodiments, the bacterium is a Gram-negative bacterium. In certain exemplary embodiments, the bacterium is E. coli or P. fluorescens.

In certain exemplary embodiments, the composition further comprises a pharmaceutical carrier or excipient. In certain exemplary embodiments, the composition is encapsulated by a pharmaceutical grade capsule in a dry powdered form. In certain exemplary embodiments, the composition is an orally-available composition.

In another aspect, a method for producing an anthelmintic composition is provided, the method comprising: (a) exposing a non-sporulating bacterium to an antimicrobial agent, thereby killing or inactivating the bacterium, wherein the bacterium is genetically engineered to express Cry14Ab and wherein the bacterium has a genetic mutation such that Cry14Ab is trapped in the cytosol of the bacterium.

In certain exemplary embodiments, the genetic mutation results in a defect of sporulation.

In certain exemplary embodiments, the method further comprises formulating the killed or inactivated bacterium in an orally-available dosage form. In certain exemplary embodiments, formulating comprises one or more of: (a) lyophilizing or spray drying the bacterium; or (b) encapsulating the bacterium in a pharmaceutical-grade capsule.

In certain exemplary embodiments, the antimicrobial agent is selected from the group consisting of: an antimicrobial compound and gamma irradiation.

In certain exemplary embodiments, the antimicrobial agent is: (a) a food-grade antibiotic; (b) a beta-lactam antibiotic; or (c) a terpene, iodine or formaldehyde. In certain exemplary embodiments, the terpene is selected from the group consisting of thymol, eugenol, geraniol, carvacrol, and citral, and combinations thereof. In certain exemplary embodiments, the terpene is carvacrol.

In certain exemplary embodiments, CryAb1 expression is under control of a non-sporulation specific promoter. In certain exemplary embodiments, the promoter is a Cry3A, GerA, GNAT, or TadA promoter.

In certain exemplary embodiments, the inactivated bacterium is Bacillus sp. In certain exemplary embodiments, the inactivated bacterium is Bacillus thuringiensis (Bt).

In certain exemplary embodiments, the genetic mutation is a deletion or inactivation of one or more genes resulting in a defect of sporulation; wherein the one or more genes is selected from the group consisting of: kinA, kinB, spo0A, spo0B, spo0E, spo0F, spo0J, spo0M spoIIB, spoIID, spoIIE, spoIIF, spoIIG, spoIIL, spoIIM, spoIIIA, spoIIIB, spoIIIE, spoIVA, spoIVC, spoIVD, spoVG, spoVK, spoVL, spoVM, spoVN, spoVP, spoVQ, spoVID, σH, σF, σE, σG, and σK. In certain exemplary embodiments, the genetic mutation is a deletion or inactivation of the spo0A gene.

In certain exemplary embodiments, the bacterium is a Gram-negative bacterium. In certain exemplary embodiments, the bacterium is an E. coli or P. fluorescens species.

In yet another aspect, a method of treating a parasitic worm infection in domesticated animals and humans is provided comprising orally administering an effective amount of the composition described above to a domestic animal or human infected with a parasitic worm.

In certain exemplary embodiments, the parasitic worm infecting the domestic animal or human is resistant to one or more other anthelmintic treatment.

In certain exemplary embodiments, the parasitic worm infecting the domestic animal or human is resistant to Cry5B.

In certain exemplary embodiments, the parasitic worm infecting the human is selected from roundworm, whipworm, hookworm, flatworm, tapeworm, flukes, and pinworms (threadworms).

In certain exemplary embodiments, the domestic animals treated are cattle, sheep, goats, equines, pigs, poultry, dogs or cats.

In certain exemplary embodiments, the parasitic worm infecting the domestic animal is selected from the group consisting of roundworm, hookworm, whipworm, heartworm, lungworm, and a strongyle (cyathostomin).

In certain exemplary embodiments, the domestic animals are sheep and the parasitic worm is a roundworm. In certain exemplary embodiments, the roundworm infecting sheep is Haemonchus contortus.

In certain exemplary embodiments, the domestic animals are equines and the parasitic worm is a strongyle (cyathostomin). In certain exemplary embodiments, the domestic animals are pigs and the parasitic worm is a roundworm.

In certain exemplary embodiments, the roundworm infecting pigs is Ascaris spp. In certain exemplary embodiments, the roundworm infection pigs is Ascaris suum.

In certain exemplary embodiments, the parasitic worm infecting the human is Ancylostoma spp or Necator spp. In certain exemplary embodiments, the parasitic worm infecting the human is Ancylostoma ceylanicum or Ancylostoma duodenale. In certain exemplary embodiments, the parasitic worm infecting the human is Necator americanus. In certain exemplary embodiments, the parasitic worm infecting the human is Ascaris spp. In certain exemplary embodiments, the parasitic worm infecting the human is Ascaris lumbricoides. In certain exemplary embodiments, the parasitic worm infecting the human is Thrichuris spp. In certain exemplary embodiments, the parasitic worm infection the human is Thrichuris trichiura.

In one aspect, an animal feed composition is provided comprising: (a) a base animal feed, and (b) a supplement comprising the composition described above.

In another aspect, a method of controlling or preventing parasitic worm infections in domestic animals is provided comprising the use of the animal feed composition described above to feed the domestic animals.

In yet another aspect, a method promoting gut health, boosting immunity, or preserving feedstuff in storage comprising use of the animal feed composition described above to feed the domestic animals.

In one aspect, a composition for human or animal waste treatment is provided, comprising the composition described in certain embodiments above.

In one aspect, a method of waste treatment is provided, comprising the step of treating waste with a composition comprising the composition described above.

In certain exemplary embodiments, the step of treating the waste comprises administering the composition to one or more of the following: waste, litter, and bedding.

In one aspect, an orally-available composition is provided comprising a killed, non-sporulating bacterium that expresses Cry14Ab and one or more additional nematicidal proteins, wherein the bacterium has a genetic mutation comprising a deletion or inactivation of one or more genes selected from the group consisting of kinA, kinB, spo0A, spo0B, spo0E, spo0F, spo0J, spo0M, spoIIB, spoIID, spoIIE, spoIIF, spoIIG, spoIIL, spoIIM, spoIIIA, spoIIIB, spoIIIE, spoIVA, spoIVC, spoIVD, spoVG, spoVK, spoVL, spoVM, spoVN, spoVP, spoVQ, spoVID, σH, σF, σE, σG, and σK that results in a defect in sporulation, wherein the Cry14Ab and the one or more additional nematicidal proteins are trapped in the cytosol of the bacterium.

In certain exemplary embodiments, the one or more additional nematicidal protein is Cry5B.

In another aspect, a method is provided for producing the composition described above, the method comprising: (a) exposing a non-sporulating bacterium to an antimicrobial agent, thereby killing or inactivating the bacterium, wherein the bacterium is genetically engineered to express Cry14Ab and one or more additional nematicidal proteins, wherein the bacterium has a genetic mutation that prevents sporulation such that Cry14Ab and the one or more nematicidal proteins are trapped in the cytosol of the bacterium; and optionally, (b) formulating the killed or inactivated bacterium in an orally-available dosage form.

In one aspect, a method of treating a parasitic worm infection in domesticated animals and humans is provided, comprising orally administering an effective amount of the composition described above to a domestic animal or human infected with a parasitic worm.

In one aspect, an animal feed composition is provided, comprising: (a) a base animal feed, and (b) a supplement comprising the composition described above.

In one aspect, a method of controlling or preventing parasitic worm infections in domestic animals is provided comprising the use of the animal feed composition described above to feed the domestic animals.

In one aspect, a method of promoting gut health, boosting immunity, or preserving feedstuff in storage is provided comprising administering or using the animal feed composition described above.

In one aspect, a composition for human or animal waste treatment is provided, comprising the composition described above.

In another aspect, a method of waste treatment is provided, comprising the step of treating waste with a composition described above.

In certain exemplary embodiments, the step of treating the waste comprises administering the composition described above to one or more of the following: waste, litter, and bedding.

### BRIEF DESCRIPTION OF THE DRAWINGS

The foregoing and other features and advantages of the present invention will be more fully understood from the following detailed description of illustrative embodiments taken in conjunction with the accompanying drawings. The file of this patent contains at least one drawing/photograph executed in color. Copies of this patent with color drawing(s)/photograph(s) will be provided by the Office upon request and payment of the necessary fee.

FIG. 1A-FIG. 1B depict inhibition of larval development from eggs of Haemonchus contortus (FIG. 1A) and Cyathostomins (FIG. 1B) by bacteria carrying an empty vector (KFBT-43 (EV)) or a vector expressing Cry14Ab (KFBT-72). Amount of bacteria added varied from 0 to 1100 ng/ml. Error bars represent standard errors.

FIG. 2A-FIG. 2B depict in vivo effects of Cry14Ab IBaCC lysate on reduction of fecal egg counts (FIG. 2A) and worm burdens (FIG. 2B) of H. contortus in sheep.

FIG. 3 depicts the effect of Cry14Ab IBaCC lysate on the viability of Ascaris suum L4 larvae in vitro.

FIG. 4 depicts the effect of various doses of Cry14Ab IBaCC lysate on the motility of adults T. muris worms in vitro.

FIG. 5A-FIG. 5B depict the in vivo effect of a single dose of Cry14Ab IBaCC lysate on the fecal egg count (FIG. 5A) and worm burdens of A. celyanicum in hamsters (FIG. 5B).

FIG. 6A-FIG. 6B depict the effect of a single 50 mg dose of Cry14Ab IBaCC lysate on fecal egg counts (FIG. 6A) and worm burdens (FIG. 6B) of Heligmosomoides bakeri in infected mice.

FIG. 7A-FIG. 7B depict with the in vivo effects of a 50 mg/kg dose of Cry14Ab IBaCC lysate on intestinal worm burdens (FIG. 7A) of Necator americanus and fecal egg counts (FIG. 7B) and in infected hamsters.

FIG. 8A-FIG. 8B depict the motility of Ascaris suum intestinal L4 stage at two different doses of Cry14Ab IBaCC lysate over time. 3=fully motile; 2=inhibited motility; 1=immotile until touched; 0=immotile even with touch (n=20 per dose; 5 per experiments repeated 4 times) (FIG. 8A) and Ascaris suum intestinal worm burdens in mice in control (water; n=6) and Cry14Ab IBaCC lysate treated groups (n=5). Treated group: Two doses of 30 mg/kg Cry14Ab given two days in a row to infected mice (FIG. 8B).

### DETAILED DESCRIPTION

Disclosed are methods of treating or preventing STH infection by administering to a subject a preparation of killed or inactive bacteria recombinantly expressing a nematicidal protein (e.g., crystal protein from Bacillus thuringiensis) in the cytosol of the bacterium. Such recombinant bacteria are treated with an anti-microbial agent such that the bacteria are killed before or during administration. In these particular methods, because the bacteria are dead when administered, any bacterium, including non-food grade bacteria, can be administered to a subject to treat an STH infection.

### Microbes

In certain embodiments, the bacteria of the invention are non-sporulating bacteria. As used herein, the term “non-sporulating bacterium” includes wild-type bacteria that are incapable of producing spores (e.g., certain Gram-negative bacteria) as well as genetic variants of spore-forming bacteria that have been engineered to be defective in sporulation (e.g., certain Gram-positive bacteria). As used herein, unless the context makes clear otherwise, “a mutation resulting in a defect in sporulation” or “a genetic mutation that results in a defect in sporulation” refers to any genetic mutation that results in a defect in a member of the sporulation pathway and/or any genetic mutation that prevents the formation of viable spores.

In some embodiments, sporulation-deficient bacteria are advantageous. An example of a sporulation deficient bacterium is a spo0A-Bacillus thuringiensis. Any mutation or combination of mutations that confers sporulation deficiency but that does not substantially affect viability or heterologous gene expression can be used. These mutations include but are not limited to mutations in the following genes: kinA, kinB, spo0A, spo0B, spo0E, spo0F, spo0J, spo0M spoIIB, spoIID, spoIIE, spoIIF, spoIIG, spoJIL, spoIIM, spoIIIA, spoIJIB, spoIIIE, spoIVA, spoIVC, spoIVD, spoVG, spoVK, spoVL, spoVM, spoVN, spoVP, spoVQ, spoVID, σH, σF, σE, σG, and σK. (Silvaggi, J., et al. Unmasking novel sporulation genes in Bacillus subtillus. J Bacteriol. 186, 8089-8095, 2004; Sandman, K., et al. Genetic Analysis of Bacillus subtilis spo Mutations Generated by Tn917-Mediated Insertional Mutagenesis. Genetics. 117, 603-617, 1987; Malvar and Baum, Tn5401 Disruption of the spo0F Gene, Identified by Direct Chromosomal Sequencing, Results in CrylIlIA Overproduction in Bacillus thuringiensis. J Bacteriol. 176, 4750-4753, 1994)

Bacteria are particularly applicable to the control of STHs because 1) recombinant bacteria can cheaply express large amounts of Cry proteins prior to administration into the GI tract of a mammalian subject, and Cry proteins so expressed, independent of any Cry proteins that may be secreted by bacteria in the GI tract, have been shown to have a significant impact on STHs, 2) studies using purified Cry protein to treat hookworms, whipworms, and Heligmosomoides bakeri, all in infected rodents, demonstrate that STHs in the mammalian GI tract can ingest and be killed/intoxicated by Cry proteins, 3) recombinant bacteria expressing a therapeutic protein, in which the protein is not purified, are cheaper to produce since no purified protein is needed, and 4) recombinant bacteria delivering STH curing proteins (e.g., Cry5B) are more effective that purified proteins (e.g., Cry5B) at the same bio-active protein dose (e.g., total Cry5B) in curing infections.

Microbes of the disclosed compositions and methods include killed and inactivated forms of Bacillus sp., including Bacillus subtilis (e.g., Bacillus subtilis natto, and Bacillus subtilis PY79), B. cereus, (e.g., B. cereus var. Toyoi (Toyocerin), B. cereus var. toyoii), B. toyonensis, B. clausii, B. pumilus and Bacillus thuringiensis. Bacillus subtilis has been extensively characterized as a safely ingested food additive in humans (see Example 14, infra, references 15-27). In certain exemplary embodiments, killed and inactive forms of Bacillus thuringiensis are used.

Other useful bacteria include but are not limited to non-sporulating variants of Lactococcus sp., Lactobacillus sp., Bifidobacterium sp., Streptococcus sp., Clostridium sp., Sporolactobacillus sp, Sporosarcina sp., Brevibacillus sp, Leuconostoc sp., Pedicoccus sp., Enterococcus sp. and Escherichia sp. Lactococcus sp. includes but is not limited to L. lactis. Lactobacillus sp. includes but is not limited to L. casei, L. paracasei, L. acidophilus, L. bulgaricus, L. delbrueckii subsp. bulgaricus, L. helveticus, L. plantarum, L. salivarius, L. reuteri, L. gasseri, and L. animalis. Bifidobacterium sp. includes but is not limited to B. animalis, B. bifidum, B. breve, B. infantis, and B. longum. Streptococcus sp. includes but is not limited to S. thermophilus. Clostridium sp. includes but is not limited to Clostridium butyricum. Sporolactobacillus sp. includes but is not limited to Sporolactobacillus vineae. Sporosarcina sp. includes but is not limited to Sporosarcina pasteurii. Brevibacillus sp. includes but is not limited to Brevibacillus laterosporus.

Still other useful bacteria useful in connection with the claimed invention include killed and inactivated forms of Gram-negative bacteria. In certain exemplary embodiments, the Gram-negative bacteria include E. coli species (e.g., NISSLE 1917) and Pseudomonas species (e.g., Pseudomonas fluorescens). Exemplary Cry-expressing Gram-negative bacteria which can be killed or inactivated by the methods of the invention include the Cry-expressing E. coli strain of Ge et al. (“Hyperexpression of a Bacillus thuringiensis delta-endotoxin-encoding gene in Escherichia coli: properties of the product,” Gene, 93: 49-54 (1990)) and the P. fluorescens strain of Peng et al. (“A Delta-endotoxin encoded in Pseudomonas fluorescens displays a high degree of insecticidal activity,” App. Microbiol Biotech., (2003), 63:300-306).

### Nematicidal Proteins

As used herein, unless the context makes clear otherwise, “nematicidal protein” refers to any protein that has toxic activity against nematodes or helminths. Exemplary nematicidal proteins include Cry14Ab protein in the bacterium for delivery into a helminth (e.g., roundworm)-infected vertebrate animal gastrointestinal tract via oral dosing (gavage, drinking, eating, pill, capsule, powder, etc.). The Cry proteins are expressed in the cytosol of the bacterium, allowing access to the anthelmintic protein after the bacterium lyses or opens up either due to digestion within the gastrointestinal tract ingestion and digestion of bacteria by the parasitic helminths (e.g., roundworms such as hookworms, whipworms, Ascaris, Strongyloides, veterinary parasitic roundworms of the intestine), etc.

In certain embodiments, a bacterium as provided herein may be introduced that expresses an individual Cry protein or that simultaneously expresses multiple Cry proteins. In some embodiments, multiple bacteria may be introduced, each of which expresses either a different individual Cry protein or simultaneously expresses multiple Cry proteins. In these and related embodiments, it is contemplated that the GI tract may be seeded with bacteria that express either one Cry protein or multiple Cry proteins at the same time. For example, due to the lack of cross-resistance between Cry5B-resistant roundworms and Cry21A-resistant roundworms, simultaneous administration of Cry5B and Cry21A in the gastrointestinal tract may inhibit the development of parasite resistance to the combination therapy.

In the long run, removing antibiotic selection capability (e.g., genetic selection markers) from the plasmids that are used to introduce heterologous Cry protein-encoding sequences, as well as using bacterial strains that are unable to replicate outside the vertebrate host, may be desirable in order to environmentally contain the genetically modified bacteria. For example, LAB (Lactic Acid Bacteria) have been engineered to be autotrophic in thymidine or thymine synthesis such that they can only grow in the vertebrate intestine where thymidine or thymine is present and not in the environment where thymidine or thymine is not present. See, e.g., Steidler L, et al. “Biological containment of genetically modified Lactococcus lactis for intestinal delivery of human interleukin 10.” Nat Biotechnol 21: 785-789 (2003).

Cry-transformed bacteria such as Bacilli or LAB may be cultured and expression of intracellular, membrane-anchored, or secreted Cry protein by such bacteria may be confirmed using antibodies raised against each Cry protein and standard Western blotting or ELISA techniques.

To assess the bioactivity of all constructs, recombinant expressing Cry protein (full length, truncated, or variants) may be fed to the free-living nematode, C. elegans. Cry protein toxicity on C. elegans using LC50, brood-size, developmental inhibition assays on solid media and in liquid wells may then be quantitated. C. elegans can access the Cry proteins either via protein secreted onto the solid media/into the liquid well or by their ability to grind, open and digest bacteria. Confirmation that the recombinant bacteria are making bioactive Cry proteins may be obtained. Furthermore, the bioactivity (e.g., LC₅₀ in g/mL) may be quantified and the constructs giving the highest activity determined.

A survey of public database revealed a Bacillus Cry protein, Cry14Ab. Cry14Ab was shown to be active against C. elegans and shown to be somewhat unusual compared to many insecticidal Cry proteins in that the C-terminal half of the protein, commonly referred to as the crystallization region, was not easily removed in vitro by common proteases. Cry14Ab was shown to associate with and cause damage to the plant nematode intestine. Moreover, Cry14Ab expressing plants were shown to be protected from Heterodera glycines (Kahn et al. A Bacillus thuringiensis Cry protein controls soybean cyst nematode in transgenic soybean plants. Nat Commun. 2021 Jun. 7; 12(1):3380). The cloning and nucleotide sequence of Bacillus thuringiensis cry14Ab gene are described in U.S. Pat. No. 11,242,539 the disclosure of which is incorporated herein in its entirety.

### Truncations, Variants, and Sub-Variants

The crystal proteins may be truncated to enhance their effectiveness. The usefulness of Bt toxins (e.g., crystal proteins) for controlling STHs may be limited by the protein size that STHs can ingest. Some parasitic roundworms poorly ingest proteins larger than about 40 kD. Thus, the effectiveness of any particular Bt toxin may be limited by size exclusion of proteins that STHs take in and so should be small enough to be readily absorbed by the STH gut while retaining toxic activity. A truncated toxin may be easier to express in bacteria. Producing a truncated toxin also alleviates the requirement that the target STH has the proper proteases present to correctly process full length protoxin (which is inactive) to a truncated, active toxin form. Thus, a truncated toxin is immediately available for intoxication independent of whether the proper protease processing enzymes are present in the STH target. Truncated toxin may also express at a higher level in microbes because truncated toxins are soluble and less likely to form insoluble inclusions in the cell expressing them, which could be toxic to the cell or which could make the toxin fold incorrectly. Accordingly, it is desirable to produce truncated Bt toxin fragments (e.g., crystal protein fragments). Moreover, fragments of certain Bt toxins have been tested and shown to retain toxic activity and have improved biological properties. By “truncated,” when referring to a Bt toxin protein (crystal protein) is meant a Bt toxin protein that is not full-length but retains at least 5%, 10%, 20%, 30%, 40%, 50%, 60%, 70%, 80%, or 90% or more of the toxic activity of a corresponding full-length Bt toxin protein.

“Variants” or “subvariants” of Cry proteins include polypeptides with one or more substitutions, e.g., no more than 20 substitutions, alternatively no more than 10 substitutions, or substitutions at 10% or fewer of the residues, relative to a corresponding wild-type polypeptide or truncated version thereof. The variant, subvariant, or truncated polypeptide has at least 5%, 10%, 20%, 30%, 40%, 50%, 60%, 70%, 80%, or 90% of the activity, e.g., toxic activity, of the corresponding wild-type polypeptide or truncated version. Conservative substitutions include substitutions within the following groups: glycine, alanine, threonine, valine, isoleucine, leucine; aspartic acid, glutamic acid, asparagine, glutamine; serine, cysteine; lysine, arginine; aspartic acid, glutamic acid; serine, threonine; asparagine, glutamine; phenylalanine, tyrosine.

The crystal proteins may be full length, truncated, variants, or subvariants. The truncated crystal protein may include any truncation of the N- and C-termini that still retains toxin activity. The truncated form is not full-length but retains at least 5%, 10%, 20%, 30%, 40%, 50%, 60%, 70%, 80%, or 90% or more of the toxic activity of a corresponding full-length Bt toxin protein. For example, the truncated portion may be truncated between the end of conserved block 5 and the C-terminus of the full-length protein.

Nucleic acid molecules encoding amino acid sequence variants, truncated versions, or both, of a Cry protein are prepared by a variety of methods known in the art. These methods include, but are not limited to, isolation from a natural source (in the case of naturally occurring amino acid sequence variants) or preparation by, for example, oligonucleotide-mediated (or site-directed) mutagenesis, PCR mutagenesis, and cassette mutagenesis of an earlier prepared variant or a non-variant version of protein. Moreover, the invention includes synthetic nucleic acid molecules where nucleotides are modified to include codons preferred in a particular organism, remove codons rarely used in a particular organism, or remove sequences that may inhibit transcription or RNA processing and the like.

### Anthelmintic Experiments

Once heterologous Cry protein expression and bioactivity are confirmed in a desired bacterium, the modified bacteria may be used for curative-type and preventative-type anthelmintic experiments.

Antibody production: Antibodies against recombinant Cry proteins (e.g., Cry5B, Cry21A, Cry14A, Cry14Ab, Cry13A, and Cry6A, full length and truncated proteins) may be produced and purified according to standard methodologies (e.g., Current Protocols in Immunology, John Wiley & Sons, New York, N.Y. (2009).

Bioactivity tests: To assess the bioactivity of all constructs, recombinant bacilli or other bacteria expressing heterologous Cry proteins are fed to the free-living nematode, C. elegans. C. elegans can access the Cry proteins either via protein secreted onto the solid media/into the liquid well, by protein naturally released as bacteria break open, or by their ability to grind and digest bacteria to open the bacterial cells.

Rodent and parasite tests: Three intestinal parasitic nematodes Heligmosomoides bakeri, H. polygyrus (small intestine nematode parasites) in mice, and Trichuris muris (whipworm) in mice, and A. ceylanicum (hookworm) in hamsters are tested. The tests address: 1) where in the GI tract do heterologous Cry-expressing bacteria reside and for how long; and 2) how do these bacteria affect the acquisition and progression of intestinal nematode parasites.

Parasite tests: Naïve (uninfected) mice are gavaged with the best heterologous Cry-protein expressing recombinant bacterial strain(s) based on expression and bioactivity. Protect against progression test: Mice are infected with H. bakeri. Two weeks later, infected mice are treated with heterologous Cry-protein expressing or control bacteria, respectively. Intestinal worm burdens and fecal egg counts are used to determine if the recombinant bacteria provide anthelmintic therapy in mice with pre-existing nematode infections.

### Exemplary Parasites

The disclosed methods relate to the control of parasitic worms, e.g., nematodes and platyhelminths, using crystal proteins from Bacillus and their derivatives. Parasitic worms within the scope of the invention include but are not limited to those in Class Adenophorea, e.g., Order Mononchida, Family Plectidae, and Order Stichosomida, Family Mermithidae and Tetradonematidae; Class Secernentea, e.g., Order Rhabditida, Family Carabonematidae, Cephalobidae, Chambersiellidae, Heterorhabditidae, Oxyuridae, Panagrolaimidae, Rhabditidae, Steinernematidae, Syrphonematidae, Syrphonematidae, or Thelastomatidae; Order Spirurida, Family Filariidae, Onchocercidae, Physalopteridae, Syngamidae, Spiruridae, Subuluridae, or Thelaziidae; Order Diplogasterida, Family Diplogasteridae; and Order Tylenchida, Family Allantonematidae, Aphelenchidae, Aphelenchoididae, Entaphelenchidae, Fergusobiidae, Phaenopsitylenchidae, Sphaerulariidae, Anguinidae, Dolichodoridae, Belonolaimidae, Pratylenchidae, Hoplolamidae, Heteroderidae, Criconematidae, Tylenchulidae or Tylenehidae. In one embodiment, the parasite is from Class Secernentea, Order Ascaridida, Family Ascarididae; Class Adenophorea, Order Trichurida, Family Trichuridae; Class Secernentea, Order Strongylida, Family Ancylostomatidae (ancylostomidae) or Trichostrongylidae; or Class Secernentea, Order Spirurida, Family Dracunculidae, Filariidae, or Onchocercidae.

The parasite may be a helminth. Helminths within the scope of the invention include but are not limited to those from Phylum Annelida, Class Polychaetae, Class Myzostomida, Class Clitellata, Subclass Hirudinea, Order Gnathobdellidae, Order Rhynchobdellidae; Phylum Platyhelminthes (Flatworms), Class Turbellaria, Class Monogenea, Order Monopisthocotylea, Order Polyopisthocotylea, Class Trematoda, Subclass Aspidogasrea, Subclass Digenea; Super Order Anepitheliocystida, Order Strigeatida, Family Schistosomatidae, Subfamily Schistosomatinae, Genus Schistosoma, Order Echinostomatida, Family Fasciolidae, Family Paramphistomatidae, Family Echinostomatidae; Super Order Epitheliocystida, Order Plagiorchiida, Family Dicrocoeliidae, Family Troglotrematidae, Order Opisthorchiida, Family Heterophyidae, Family Opisthorchiidae, Class Cestoda, Subclass Cestodaria, Subclass Eucestoda, Order Pseudophyllidea, Family Diphyllobothriidae, Order Cyclophyllidea, Family Taeniidae, Family Hymenolepididae, Family Dilepididae, Family Mesocestoididae, Order Tetraphyllidea, Order Proteocephalata, or Order Spatheobothridea. For example, Cry proteins with the scope of the invention may be employed to prevent, inhibit, or treat Roundworm, Whipworm, Hookworm, Schistosome, or Trematodes.

The parasite may also be gastrointestinal tract parasitic roundworms/nematodes. The gastrointestinal tract parasitic roundworms/nematodes may include but are not limited to the following species: Haemonochus, Cooperia, Ostertagia, Trichostrongylus, Teladorsagia, Nematodirus, Ancylostoma, Cyathostominea/Cyathostomin/Cyathostome, Strongylus, Parascaris, Ascaris, Trichuris, Oesophagostomum/Oesophagustomum, Trichiuris, Bunostomum, Oxyuris, Chabertia, Habronema, Draschia, Triodontophorus, Toxocara, Toxascaris, and Uncinaria. Haemonochus species includes but is not limited to Haemonchus contortus and Haemonchus placei, Cooperia species includes but is not limited to Cooperia oncophora, Cooperia pectinata, and Cooperia curticei. Ostertagia species includes but is not limited to Ostertagia ostertagi, Ostertagia (Teladorsagia) circumcincta, and Ostertagia trifurcate. Trichostrongylus species includes but is not limited to Trichostrongylus axei, Trichostrongylus colubriformis, and T. circumcincta. Teladorsagia species includes but is not limited to Teladorsagia (Ostertagia) circumcincta. Nematodirus species includes but is not limited to Nematodirus spathiger. Ancylostoma species includes but is not limited to Ancylostoma caninum, Ancylostoma braziliense, and Ancylostoma tubaeforme. Cyathostominea/Cyathostomin/Cyathostome nematodes are also included. Strongylus species (small and large) includes but is not limited to Strongylus vulgaris, Strongylus equinus, and Strongylus edentatus. Parascaris species includes but is not limited to Parascaris equorum. Strongyloides species includes but is not limited to Strongyloides westeri. Ascaris species includes but is not limited to Ascaris suum. Trichuris species includes but is not limited to Trichuris globulosa, Trichuris suis, Trichuris campanula, and Trichuris vulpis. Oesophagostomum/Oesophagustomum species includes but is not limited to Oesophagustomum dentatum, Oesophagustomum quadrispinulatum, Oesophagostomum columbianum, and Oesophagostomum venulosum. Trichiuris species includes but is not limited to Trichiuris ovis. Bunostomum species includes but is not limited to Bunostomum trigonocephalum. Oxyuris species includes but is not limited to Oxyuris equi (pin worms). Chabertia species includes but is not limited to Chabertia ovina. Habronema species includes but is not limited to Habronema microstoma and Habronema muscae. Draschia species includes but is not limited to Draschia megastoma. Triodontophorus species includes but is not limited to Triodontophorus minor and Triodontophorus serrates. Toxocara species includes but is not limited to Toxocara canis and Toxocara cati. Toxascaris species includes but is not limited to Toxascaris leonine. Uncinaria species includes but is not limited to Uncinaria stenocephala. Human parasitic roundworms of the gastrointestinal tract include but are not limited to the hookworms Ancylostoma duodenale and Necator americanus, the whipworm Trichuris trichiura, the roundworm Ascaris lumbricoides, the threadworm Strongyloides stercoralis, and the pinworm Enterobius vermiculari.

### Anti-Microbial Agents

In the disclosed methods, the recombinant bacteria expressing a crystal protein can be treated with an anti-microbial agent. Anti-microbial agents can be used on the recombinant bacteria before administration to a subject, or concomitant with administration to the subject. An advantage of killing the recombinant bacteria is that otherwise non-food safe bacteria can be used in the disclosed methods. Such non-food safe bacteria, such as Bacillus thuringiensis which is closely related to Bacillus cereus that can cause food poisoning, express very high levels of Cry proteins such as Cry5B and improve the efficacy of the protein when co-administered versus when the protein is administered in a pure form without the bacterium

Suitable anti-microbial agents are those that (1) sufficiently kill the recombinant bacteria; and (2) do not substantially affect the activity and/or levels of the crystal protein. Examples of suitable anti-microbial agents include, but are not limited to, antibiotics (such as a beta-lactam antibiotic), bactericidal agents, iodine, terpenes, formaldehyde, and irradiation. Examples of terpenes include, but are not limited to, thymol, eugenol, geraniol, carvacrol, and citral, or a combination thereof. Carvacrol is especially useful.

### Additional Therapeutic Agents

In certain embodiments the crystal protein-recombinant bacteria are administered in combination with at least one additional therapeutic agent. This additional agent can be, for example, a bacterium expressing or capable of expressing, a crystal protein, a small molecule, or a polypeptide (including antibodies and fragments thereof). In a further embodiment, the additional therapeutic is a nicotinic acetylcholine receptor agonist. In certain embodiments, the additional therapeutic agent is administered simultaneously with recombinant bacteria. In certain embodiments the additional therapeutic agent is administered sequentially (and in either order) with the recombinant bacterium. In certain embodiments, the nicotinic acetylcholine receptor agonist is from the levamisole family of nicotinic acetylcholine receptor agonists. In certain embodiments, the nicotinic acetylcholine receptor agonist is levamisole. In certain embodiments, the levamisole is administered in an amount of about 0.1 mg/kg to about 5.0 mg/kg. In certain embodiments the nicotinic acetylcholine receptor agonist is pyrantel or tribendimidine. In certain embodiments, the pyrantel is administered in an amount of about 1.0 mg/kg to about 15.0 mg/kg. In certain embodiments, the tribendimidine is administered in an amount of about 0.25 mg/kg to about 10 mg/kg.

### Administration, Dosage Forms, Pharmaceutical Compositions

The present invention describes compositions and methods for administration of killed or inactivated bacterial cells to the gastrointestinal tract of a subject. The methods include administering the bacteria in food or as a food supplement. Oral administration is preferably in an aqueous suspension, emulsion, powder or solid. The composition may be formulated into a food or added to food by the user prior to consumption. Administration to the gastrointestinal tract may also be in the form of an anal suppository (e.g., in a gel or semi-solid formulation). All such formulations are made using standard methodologies.

The method is typically practiced on any animal where inhibiting pathogen or parasites is desired. In certain embodiment, the animal is a human. However, the animal can be any livestock or zoological specimen where such inhibition of parasites/pathogens provides economic and health benefits. Any animal can benefit by the claimed methods, including birds, reptiles, mammals such as horses, cows, sheep, goats, pigs, and the like domesticated animals, or any of a variety of animals of zoological interest. Other purposes are readily apparent to one skilled in the arts of nutrient absorption, feed utilization and bioavailability.

The present invention further contemplates a therapeutic system for treating, reducing and/or controlling parasitic infections. Typically, the system is in the form of a package containing a therapeutic composition of the present invention, or in combination with packaging material. The packaging material includes a label or instructions for use of the components of the package. The instructions indicate the contemplated use of the packaged component as described herein for the methods or compositions of the invention. By way of example, and not of limitation, a system can comprise one or more unit dosages of a therapeutic composition according to the present invention. Alternatively, the system can alternately contain bulk quantities of a therapeutic composition. The label contains instructions for using the therapeutic composition in either unit dose or in bulk forms as appropriate and may also include information regarding storage of the composition, disease indications, dosages, routes and modes of administration and the like information.

Furthermore, depending upon the particular contemplated use, the system may optionally contain either combined or in separate packages one or more of the following components: bifidogenic oligosaccharides, flavorings, carriers, and the like components. One particularly preferred embodiment comprises unit dose packages of bacterial cells for use in combination with a conventional liquid product, together with instructions for combining the bacteria with the formula for use in a therapeutic method.

Different dosage regimens may be used in the disclosed methods. In some embodiments, a daily dosage is administered once, twice, three times, or four times a day for one, two, three, four, five, six, seven, eight, nine, or ten days. In some embodiments, a once- or twice-daily dosage is administered every other day.

Administration of the compositions containing the active ingredients effective in inhibiting parasite growth in the intestine and in feces generally consist of one to ten unit dosages of 10 mg to 10 g per dosage of the composition for one day up to one month for a human of approximately 100 kg body weight. Unit dosages are generally given once every twelve hours and up to once every four hours. Preferably two to four dosages of the composition per day, each comprising about 0.1 g to 50 g per dosage, for one to seven days are sufficient to achieve the desired result.

A preferred method involves the administration into the digestive tract of from 1×10² to 1×10¹⁰ of bacterium per day, in some embodiments from 11×10³ to 1×10⁶, in other embodiments from 1×10⁶ to 1×10⁹, and more preferably about from 5×10⁸ to 1×10⁹ bacterium per day. Exemplary dosages range from about 1×10³ to 1×10⁶ bacterium per day, or alternatively range from about 1×10⁶ to 1×10⁹ bacterium per day.

In various specific embodiments, an effective dose of a composition of the present disclosure can be in a range of from 1.0 gm to 15.0 gm for an adult patient, more preferably between about 2.0 gm and about 10.0 gm of the composition. Effective doses can be administered to a subject at any suitable frequency, e.g., at least once a week, preferably once a day. Pediatric dosages may be in the range of 15% to 90% of adult dosages.

In other embodiments, a constant dosage of the composition can be administered over time, for example about 2 gm to about 4 gm per day, up to about 6 g to about 10 g per day, depending on the severity of the physiological condition. Once the infection has been effectively ameliorated, the subject can in many instances decrease the dosage to about 2 gm to about 4 gm per day for maintenance purposes. The desired dose may be presented in multiple (e.g., two, three, four, five, six, or more) sub-doses administered at appropriate intervals throughout the day.

The pharmaceutical compositions comprising the crystal protein-expressing recombinant bacteria can be administered via any of the accepted modes of administration or agents known in the art. However, oral administration is preferred because this route of delivery delivers the recombinant bacteria to the GI tract. The dosage form can be, for example, a solid, semi-solid, lyophilized powder, or liquid dosage forms, such as for example, tablets, pills, soft elastic or hard gelatin capsules, powders, solutions, suspensions, suppositories, aerosols, or the like, and can be in unit dosage forms suitable for simple administration of precise dosages. One exemplary embodiment of the dose form is a capsule containing the composition of the disclosure including the bacterial species in a dried form, blended with pharmaceutical carrier. The capsule for such dose form can be of any suitable type, e.g., a gelatin capsule of a conventional variety.

The physiologically compatible carrier medium with which the bacterial species are employed, can be of any simple type, e.g., a pharmaceutically acceptable carrier such as fructo-oligo-saccharide (FOS) medium, or other soluble fiber, sugar, nutrient or base material for the composition, with which the bacterial species can be formulated, e.g., in an orally administrable form. Other carrier media include mannitol, inulin (a polysaccharide), polydextrose, arabinogalactan, polyolslactulose, lactitol, etc. A wide variety of materials can be used as carrier material in the practice of the present disclosure, as will be apparent to those of ordinary skill in the art, based on the description herein.

The carrier medium, when present, can be blended with the bacterial species in any suitable amounts, such as an amount of from 5% to 95% by weight of carrier medium, based on the total weight of the bacterial species and the carrier medium, in various embodiments.

In other embodiments, the amount of carrier medium may be in a range having a lower limit of any of 5%, 10%, 12%, 15%, 20%, 25%, 28%, 30%, 40%, 50%, 60%, 70% or 75%, and an upper limit, higher than the lower limit, of any of 20%, 22%, 25%, 28%, 30%, 40%, 50%, 60%, 70%, 75%, 80%, 85%, 90%, and 95%. The amount of carrier medium in a specific embodiment may be determined based on considerations of the specific dose form, relative amounts of the bacterial species, the total weight of the composition including the carrier medium and the bacterial species, and the physical and chemical properties of the carrier medium, and other factors, as known to those of ordinary skill in the probiotic formulation art.

In certain embodiments, the bacterial cells are formulated in a composition that protects the cells and/or Cry proteins from the acid environment of the stomach. Accordingly, the invention includes a composition containing a bacterium and a pharmaceutically acceptable acid-resistant (“enteric”) carrier. By acid-resistant is meant that the carrier or coating does not dissolve in an acidic environment. An acidic environment is characterized by a pH of less than 7. The acid-resistant carrier is resistant to acids at pH less than about 4.0. Preferably, the carrier does not dissolve in pH 2-3. Most preferably, it does not dissolve in pH of less than 2. To protect bacterial cells from stomach acids, the cells are coated or encapsulated with the acid-resistant carrier.

In certain embodiments, the coating is pH sensitive. For example, the coating may dissolve after the pH is greater than 4.0. For example, the coating dissolves in a neutral environment as is encountered in the small intestine and does not dissolve in an acidic environment as is encountered in the stomach. Alternatively, the enteric coating dissolves when exposed to specific metabolic event such as an encounter with a digestive enzyme that is found in the small intestine. For example, the coating is digested by a pancreatic enzyme such as trypsin, chymotrypsin, or a pancreatic lipase. The formulation is hydrated in the small intestine. Digestion or dissolution of the coating allows liberation of bacterial cells, e.g., Bacillus cells, into the intestine.

In other embodiments, bacterial cells are stabilized in a gel or paste such as an anhydrous carbohydrate paste. In alternate formulations, the cells are lyophilized and/or suspended in a gel or paste. Enteric coating materials are known in the art, e.g., malic acid-propane 1,2-diol. Cellulose derivatives, e.g., cellulose acetate phthalate or hydroxypropyl methylcellulose phthalate (HPMCP), are also useful in enteric acid-resistant coatings. Other suitable enteric coatings include cellulose acetate phthalate, polyvinyl acetate phthalate, hydroxypropylmethylcellulose phthalate and anionic polymers of methacrylic acid and methyl methacrylate. Another suitable enteric coating is a water emulsion of ethylacrylate methylacrylic acid copolymer, or hydroxypropyl methyl cellulose acetate succinate (HPMAS). (See, e.g., U.S. Pat. No. 5,591,433). An enteric coating is designed to resist solution in the stomach and to dissolve in the neutral or alkaline intestinal fluid. In certain embodiments, the bacterial cells are preferably formed into dry powders. Suitable drying methods include a natural drying, a forced-air drying, a spray drying, a freeze drying, and the like. Of those, a spray drying, drum drying or a forced-air drying are preferably used. A protective agent such as skim milk, sodium glutamate, and saccharides may be used in a time of drying. As saccharides, glucose and trehalose may be used.

Auxiliary and adjuvant agents may include, for example, preserving, wetting, suspending, sweetening, flavoring, perfuming, emulsifying, and dispensing agents. Prevention of the action of contaminating microorganisms; if desired, can be accomplished using various antibacterial and antifungal agents, such as, parabens, chlorobutanol, phenol, sorbic acid, and the like. Isotonic agents, such as sugars, sodium chloride, and the like, may also be included.

Solid dosage forms can be prepared with coatings and shells, such as enteric coatings and others well-known in the art. They can contain pacifying agents and can be of such composition that they release the active compound or compounds in a certain part of the intestinal tract in a delayed manner. Examples of embedded compositions that can be used are polymeric substances and waxes. The active compounds also can be in microencapsulated form, if appropriate, with one or more of the above-mentioned excipients.

Liquid dosage forms for oral administration include pharmaceutically acceptable emulsions, solutions, suspensions, syrups, and elixirs. Such dosage forms are prepared, for example, by dissolving, dispersing, etc., the active agent (such as the recombinant bacteria), and optional pharmaceutical adjuvants in a carrier, such as, for example, water, saline, aqueous dextrose, glycerol, ethanol and the like; solubilizing agents and emulsifiers, as for example, ethyl alcohol, isopropyl alcohol, ethyl carbonate, ethyl acetate, benzyl alcohol, benzyl benzoate, propyleneglycol, 1,3-butyleneglycol, dimethyl formamide; oils, in particular, cottonseed oil, groundnut oil, corn germ oil, olive oil, castor oil and sesame oil, glycerol, tetrahydrofurfuryl alcohol, polyethyleneglycols and fatty acid esters of sorbitan; or mixtures of these substances, and the like, to thereby form a solution or suspension.

Actual methods of preparing such dosage forms are known, or will be apparent, to those skilled in this art. Reference is made, for example, to Remington's Pharmaceutical Sciences, 18th ed. (Mack Publishing Company, Easton, Pa., 1990).

### Methods

The methods are directed to treating a parasitic worm or helminth infection in a subject comprising administering to the subject a therapeutically effective amount of a composition comprising an anti-microbial agent treated recombinant bacterium that is engineered to express a crystal protein.

Furthermore, the methods are directed to reducing the severity of a parasitic worm or helminth infection comprising administering to the subject a therapeutically effective amount of a composition comprising an anti-microbial agent treated recombinant bacterium that is engineered to express a crystal protein.

Methods for the expression of crystal proteins in the IBaCC (inactivated bacteria with crystal proteins) are described in detail in U.S. Pat. No. 11,484,568 and in Li et al. (Antimicrob Agents Chemother. 2021 Feb. 17; 65 (3):e01469-20). The disclosures of both of these references are incorporated herein in their entirety.

### Selected Definitions

As used herein, unless the context makes clear otherwise, “treatment,” and similar words such as “treated,” “treating” etc., indicates an approach for obtaining beneficial or desired results, including and preferably clinically desirable results. Treatment can involve optionally either the amelioration of symptoms of the disease or condition, or the delaying of the progression of the disease or condition.

As used herein, unless the context makes clear otherwise, “subject” means a vertebrate, such as a mammal. The mammal can be a feline, a rodent, a canine, a bovine, an equine, a swine, a caprine, an ovine, or a primate. In some embodiments, the subject is a human.

As used herein, unless the context makes clear otherwise, “reducing the likelihood of occurrence,” “prevention,” and similar words such as “prevented,” “preventing” etc., include approaches for preventing, inhibiting, or decreasing the likelihood of the onset or recurrence of a disease or condition, in a manner that exhibits statistical significance, for example, when compared to the results obtained when the indicated method steps are omitted. Similarly, also included are preventing, inhibiting, or decreasing the likelihood of the occurrence or recurrence of the symptoms of a disease or condition, or optionally delaying the onset or recurrence of a disease or condition, or delaying the occurrence or recurrence of the symptoms of a disease or condition. As used herein, “prevention” and similar words also include reducing the intensity, effect, symptoms and/or burden of a disease or condition prior to onset or recurrence of the disease or condition. Methods according to these and related embodiments may be practiced using an effective amount or a therapeutically effective amount of an agent that substantially eradicates, reduces the severity of, or reduces the likelihood of occurrence of a soil-transmitted helminth (STH) infection. As used herein, an “effective amount” or a “therapeutically effective amount” of a composition, agent or substance is that amount sufficient to obtain a desired biological effect, such as beneficial results, including clinical results.

As used herein “IBaCC” means inactivated bacteria with cytosolic crystals rendered inviable with food-grade essential oils.

In certain preferred embodiments, the herein described compositions for treating or reducing the severity or likelihood of occurrence of an STH infection are formulated as pharmaceutical compositions, which will preferably be formulated for oral delivery. Pharmaceutical compositions are formulated so as to allow the agent(s) contained therein to be bioavailable upon administration of the composition to a human.

It will be appreciated that the practice of the several embodiments of the present invention will use, unless indicated specifically to the contrary, conventional methods in virology, immunology, microbiology, molecular biology, and recombinant DNA techniques that are within the skill of the art, and many of which are described below for the purpose of illustration. Such techniques are explained fully in the literature. See, e.g., Current Protocols in Molecular Biology or Current Protocols in Immunology, John Wiley & Sons, New York, N.Y. (2009); Ausubel et al., Short Protocols in Molecular Biology, 3^{rd} ed., Wiley & Sons, 1995; Sambrook and Russell, Molecular Cloning: A Laboratory Manual (3rd Edition, 2001); Maniatis et al. Molecular Cloning: A Laboratory Manual (1982); DNA Cloning: A Practical Approach, vol. I & II (D. Glover, ed.); Oligonucleotide Synthesis (N. Gait, ed., 1984); Nucleic Acid Hybridization (B. Hames & S. Higgins, eds., 1985); Transcription and Translation (B. Hames & S. Higgins, eds., 1984); Animal Cell Culture (R. Freshney, ed., 1986); Perbal, A Practical Guide to Molecular Cloning (1984) and other like references.

Standard techniques may be used for recombinant DNA, oligonucleotide synthesis, and tissue culture and transformation (e.g., electroporation, lipofection). Enzymatic reactions and purification techniques may be performed according to manufacturer's specifications or as commonly accomplished in the art or as described herein. These and related techniques and procedures may be generally performed according to conventional methods well known in the art and as described in various general and more specific references that are cited and discussed throughout the present specification. Unless specific definitions are provided, the nomenclature utilized in connection with, and the laboratory procedures and techniques of, molecular biology, analytical chemistry, synthetic organic chemistry, and medicinal and pharmaceutical chemistry described herein are those well-known and commonly used in the art. Standard techniques may be used for recombinant technology, molecular biological, microbiological, chemical syntheses, chemical analyses, pharmaceutical preparation, formulation, and delivery, and treatment of patients.

As used in this specification and the appended claims, the singular forms “a,” “an” and “the” include plural references unless the content clearly dictates otherwise. Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers. Each embodiment in this specification is to be applied mutatis mutandis to every other embodiment unless expressly stated otherwise.

### EXAMPLES

The following Examples are presented by way of illustration and not limitation.

### Example 1: Expression of Cry Proteins in Bacillus

Bacillus thuringiensis, Bacillus cereus (e.g., var. toyoi, var. toyoii), Bacillus toyonensis, Bacillus thuringiensis (e.g., var. HD1), or Bacillus subtilis (e.g., var. PY79, var. natto) is used to express Cry proteins using either sporulation promoters (stationary phase/early sporulation Cry3A, late sporulation Cry5B) or a constitutive promoter (e.g., the mbg promoter). See, e.g., Shao X, et al. “Surface display of heterologous proteins in Bacillus thuringiensis using a peptidoglycan hydrolase anchor.” MICROB CELL FACT 8: 48 (2009). These constructs are transformed into B. cereus, B. toyonensis, B. thuringiensis, and B. subtilis strains and are tested for expression and bioactivity as described below. In, addition, strong expression promoters (constitutive and inducible) have been made for B. subtilis, and these and other genetic elements described herein are referred to as being “operably linked” when they are present in a polynucleotide construct and situated in a manner that permits them to exert the desired function, such as promotion of specific gene transcription (See, e.g. Phan T T, et al. “Novel plasmid-based expression vectors for intra- and extracellular production of recombinant proteins in Bacillus subtilis.” PROTEIN EXPR PURIF 46: 189-195 (2006). Secreted versions of proteins are made by addition of the signal peptide of the amyQ gene. See id. Thus, similar expression/curative experiments are carried out using Bacillus subtilis as the probiotic strain.

### Example 2: Curative Experiment A—Protocol for Infections, Anthelmintic Treatment, and Determination of Treatment Efficacy (Small Intestine Roundworm Parasite)

Six week old female Swiss Webster mice are infected per os with a suspension of 200±Heligmosomoides bakeri infective third-stage larvae in 0.1 mL of distilled water. The outbred strain Swiss Webster is used to better “mimic” treating a genetically diverse host (like humans). Each mouse is gavaged on day 15 post-infection (PI) with 0.1 mL of buffer, 0.1 mL of high dose sporulation-defective bacterium such as spo0A-Bacilli (alive or dead) control (transformed with empty vector) or 0.1 mL of high dose bacteria expressing Cry protein (6-10 animals/group). Progression of the infection is determined by fecal egg counts every other day beginning 3 days before treatment. Mice are placed individually in empty plastic cages for 1 h each morning, and the fecal pellets are collected into 50 mL centrifuge tubes. The number of eggs present is counted using the modified McMaster technique. See Hu Y, et al. “Bacillus thuringiensis Cry5B protein is highly efficacious as a single-dose therapy against an intestinal roundworm infection in mice.” PLoS NEGL TROP Dis 4: e614 (2010). At 1, 2, or 3 weeks after treatment, the animals from all three groups are euthanized and the intestinal worm burdens are counted. Using fecal egg counts and intestinal worm burdens, the ability of Cry-expressing sporulation-defective bacterium such as spo0A-Bacilli (alive or dead) to cure small intestinal roundworm infections are ascertained.

### Example 3: Curative Experiment B—Trichuris muris: Whipworm (Large Intestine Roundworm Parasite)

Twenty-one (21) 6-8 week old female AKR mice are infected per os with 200 infectious-staged T. muris eggs. Thirty (30) days post-infection, the mice are treated per os (7/group) with a single 0.1 mL dose of buffer, 0.1 mL high dose of sporulation-defective bacterium such as spo0A-Bacilli (alive or dead) control (transformed with empty vector), or 0.1 mL of high dose sporulation-defective bacterium such as spo0A-Bacilli (alive or dead) expressing Cry protein. Fecal egg counts are taken three days before treatment and then every other day until necropsy (same protocol to collect eggs as per H. bakeri). The mice are euthanized either 1, 2 or 3 weeks after treatment and worm burdens in the large intestine are determined. Using fecal egg counts and intestinal worm burdens, the ability of Cry-expressing sporulation-defective bacterium such as spo0A-Bacilli (alive or dead) to cure large intestinal roundworm infections are ascertained.

### Example 4: Curative Experiment C—Ancylostoma ceylanicum: Hookworm (Blood Feeding, Small Intestinal Roundworm Parasite)

Twenty one (21) 4-week old Syrian hamsters are infected per os with 150 infectious staged L3 A. ceylanicum hookworm larvae. Fourteen (14) days post-infection, the hamsters are treated per os with a single 0.1 mL dose of buffer, 0.1 mL high dose of sporulation-defective bacterium such as spo0A-Bacilli (alive or dead) control (transformed with empty vector), or 0.1 mL of high dose sporulation-defective bacterium such as spo0A-Bacilli (alive or dead) expressing Cry protein. Body weight, hemoglobin levels, and fecal egg counts (beginning three days before treatment) are monitored every other day until day 21, 28, or 35, at which point the animals are euthanized and worm burdens in the small intestine are determined. Using fecal egg counts, hemoglobin levels, and intestinal worm burdens, the ability of Cry-expressing sporulation-defective bacterium such as spo0A-Bacilli (alive or dead) to cure blood-feeding small intestinal roundworm infections are ascertained.

Studies of Ancylostoma ceylanicum hookworms in Syrian hamsters were carried out as previously described (Hu, Y., et al. PLoS One, 8, e70702, 2013; Hu, Y., et al. Appl Environ Microbiol, 79, 5527-5532, 2013). Briefly, 4-6 week old male hamsters were infected with 150 infectious third staged larvae per os. On day 17 post-infection, an overnight collection of stool is taken and fecal egg counts (FECs) taken the next day. The hamsters are assigned to groups based on FEC so that there is roughly the same level of infection (same average egg per gram of feces or EPG) in all groups. On day 18 post-infection, the hamsters are weighed for dosing purposes and then gavaged with treatments as described in each experiment. On day 21 post-infection, another overnight collection of stool is taken for FECs. On day 22 post-infection, the animals were euthanized. Total hookworms in the small intestine were counted and EPGs calculated.

Studies of Necator americanus hookworms in Syrian hamsters were carried out similarly with the following differences. Following subcutaneous (subQ) infection with 150 infectious third staged larvae, hamsters were injected daily with 200 μL of 4 mg/mL dexamethasone to suppress immunological responses that expel the parasites (Fujiwara, R., et al. Parasite Immunol, 28, 285-293, 2006). Treatments were conducted on day 57 post-infection and hookworm burdens/final FECs determined on day 61 post-infection.

For all experiments involving cimetidine, hamsters or mice were pre-gavaged with 200 μL of an 8.75% cimetidine solution 15 minutes prior to therapeutic treatment (Stepek, G., et al. Parasitology, 134, 103-112, 2007).

### Example 5: Preventative-Type Experiment A

Swiss Webster mice as above (6-10 each group, three groups) received either 0.1 mL buffer, 0.1 mL high dose empty vector-transformed sporulation-defective bacterium such as spo0A-Bacilli (alive or dead) without Cry protein expression, or 0.1 mL high dose vector-transformed sporulation-defective bacterium such as spo0A-Bacilli (alive or dead) with Cry protein expression. Some (about 2-21) days later, all groups of mice are then challenged with 200 H. bakeri infectious larvae as described above. Two weeks later after infection challenge, fecal egg counts are determined every other day for one to two weeks, after which time the mice are euthanized to determine intestinal roundworm burdens. Fecal egg counts and intestinal roundworm burdens are used to determine if the probiotics protected the mice against a challenge with a small intestine roundworm parasite (i.e., prevented infection).

### Example 6: Preventative-Type Experiment B

AKR mice as above (6-10 each group, three groups) receive either 0.1 mL buffer, 0.1 mL high dose empty vector-transformed sporulation-defective bacterium such as spo0A-Bacilli (alive or dead) without Cry protein expression, or 0.1 mL high dose vector-transformed sporulation-defective bacterium such as spo0A-Bacilli (alive or dead) with Cry protein expression. Some (about 2-21) days later, all groups of mice are then challenged with 200 T. muris infectious eggs as above. Thirty (30) days after infection challenge, fecal egg counts are determined every other day for one to two weeks, after which time the mice are euthanized to determine intestinal roundworm burdens. Fecal egg counts and intestinal roundworm burdens are used to determine if the probiotics protected the mice against a challenge with a large intestine roundworm parasite (i.e., prevented infection).

### Example 7: Preventative-Type Experiment C

Hamsters as above (6-10 each group, three groups) receive either 0.1 mL buffer, 0.1 mL high dose empty vector-transformed LAB without Cry protein expression, or 0.1 mL high dose vector-transformed sporulation-defective bacterium such as spo0A-Bacilli (alive or dead) with Cry protein expression. Some (about 2-21) days later, all groups of hamsters are then challenged with 150 A. ceylanicum infectious larvae as above. Two weeks after infection challenge, fecal egg counts are determined every other day for one to two weeks, after which time the hamsters are euthanized to determine intestinal roundworm burdens. Fecal egg counts and intestinal roundworm burdens are used to determine if the treatment protected the hamsters against a challenge with a small intestine blood-feeding roundworm parasite (i.e., prevented infection). In addition to experiments with rodents described above, similar experiments could be carried out with other mammals, e.g., felines, canines, bovines, equines, swine, caprines, ovines, and primates.

### Example 8: Cloning of cry14Ab in the IBaCC Expression System

The Cry14Ab sequence was found in the public database (SEQ ID NO:1). Active Cry14Ab can also have an altered N-terminal sequence (SEQ ID NO: 2). Amino acid sequences of Cry14Ab protein are listed in Table 1.

TABLE 1
Amino acid sequence of Cry14Ab

| Sequence ID | Cry14Ab amino acid sequence |
|---|---|
| SEQ ID NO: 1 | MDCNLQSQQNIPYNVLAIPVSNVNSLTDTVGDLKKAWEEFQKTG |
| | SFSLTALQQGFSASQGGTFNYLTLLQSGISLAGSFVPGGTFVAPIIN |
| | MVIGWLWPHKNKNADTENLINLIDSEIQKQLNKALLDADRNEWS |
| | SYLESIFDSSNNLNGAIVDAQWSGTVNTTNRTLRNPTESDYTNVV |
| | TNFIAADGDIANNENHIMNGNFDVAAAPYFVIGATARFAAMQSYI |
| | KFCNAWIDKVGLSDAQLTTQKANLDRTKQNMRNAILNYTQQVM |
| | KVFKDSKNMPTIGTNKFSVDTYNVYIKGMTLNVLDIVAIWPSLYP |
| | DDYTSQTALEQTRVTFSNMVGQEEGTDGSLRIYNTFDSFSYQHSP |
| | IPNNNVNLISYYNDELQNLELGVYTPPKKGSGYSYPYGFVLNYAN |
| | SKYKYGDSNDPESLGGLSTLSAPIQQVNAATQNSKYLDGEILNGI |
| | GASLPGYCTTGCSPTEPPFSCTSTANGYKASCNPSDTNQKINALYP |
| | FTQANVKGNTGKLGVLASLVSYDLNPKNVFGELDSDTNNVILKG |
| | IPAEKGYFPNNARPTVVKEWINGASAVPLDSGNTLFMTATNLTAT |
| | QYRIRIRYANPNSNTQIGVRITQNGSLISSSNLTLYSTTDMNNTLPL |
| | NVYVIGENGNYTLQDLYNTTNVLSTGDITLQITGGDQKIFIDRIEF |
| | VPTMPVPGNTNNNNGNNNGNNNPPHHVCAIAGTQQSCSGPPKFE |
| | QVSDLEKITTQVYMLFKSSPYEELALEVSSYQISQVALKVMALSD |
| | ELFCEEKNVLRKLVNKAKQLLEASNLLVGGNFETTQNWVLGTN |
| | AYINYDSFLFNGNYLSLQPASGFFTSYAYQKIDESTLKPYTRYKVS |
| | GFIGQSNQVELIISRYGKEIDKILNVPYAGPLPITADASITCCAPEIG |
| | QCDGEQSDSHFFNYSIDVGALHPELNPGIEIGLKIVQSNGYITISNL |
| | EIIEERPLTEMEIQAVNRKNQKWEREKLLECASISELLQPIINQIDSL |
| | FKDGNWYNDILPHVTYQDLKNIIIPELPKLKHWFIENLPGEYHEIE |
| | QKMKEALKYAFTQLDEKNLIHNGHFTTNLIDWQVEGDAQMKVL |
| | ENDALALQLFNWDASASQSINILEFDEDKAYKLRVYAQGSGTIQF |
| | GNCEDEAIQFNTNSFIYQEKIVYFDTPSVNLHIQSEGSEFIVSSIDLI |
| | ELSDDQ |
| SEQ ID NO: 2 | MNPNNRQQNIPYNVLAIPVSNVNSLTDTVGDLKKAWEEFQKTGS |
| (altered N- | FSLTALQQGFSASQGGTFNYLTLLQSGISLAGSFVPGGTFVAPIIN |
| terminus) | MVIGWLWPHKNKNADTENLINLIDSEIQKQLNKALLDADRNEWS |
| | SYLESIFDSSNNLNGAIVDAQWSGTVNTTNRTLRNPTESDYTNVV |
| | TNFIAADGDIANNENHIMNGNFDVAAAPYFVIGATARFAAMQSYI |
| | KFCNAWIDKVGLSDAQLTTQKANLDRTKQNMRNAILNYTQQVM |
| | KVFKDSKNMPTIGTNKFSVDTYNVYIKGMTLNVLDIVAIWPSLYP |
| | DDYTSQTALEQTRVTFSNMVGQEEGTDGSLRIYNTFDSFSYQHSP |
| | IPNNNVNLISYYNDELQNLELGVYTPPKKGSGYSYPYGFVLNYAN |
| | SKYKYGDSNDPESLGGLSTLSAPIQQVNAATQNSKYLDGEILNGI |
| | GASLPGYCTTGCSPTEPPFSCTSTANGYKASCNPSDTNQKINALYP |
| | FTQANVKGNTGKLGVLASLVSYDLNPKNVFGELDSDTNNVILKG |
| | IPAEKGYFPNNARPTVVKEWINGASAVPLDSGNTLFMTATNLTAT |
| | QYRIRIRYANPNSNTQIGVRITQNGSLISSSNLTLYSTTDMNNTLPL |
| | NVYVIGENGNYTLQDLYNTTNVLSTGDITLQITGGDQKIFIDRIEF |
| | VPTMPVPGNTNNNNGNNNGNNNPPHHVCAIAGTQQSCSGPPKFE |
| | QVSDLEKITTQVYMLFKSSPYEELALEVSSYQISQVALKVMALSD |
| | ELFCEEKNVLRKLVNKAKQLLEASNLLVGGNFETTQNWVLGTN |
| | AYINYDSFLFNGNYLSLQPASGFFTSYAYQKIDESTLKPYTRYKVS |
| | GFIGQSNQVELIISRYGKEIDKILNVPYAGPLPITADASITCCAPEIG |
| | QCDGEQSDSHFFNYSIDVGALHPELNPGIEIGLKIVQSNGYITISNL |
| | EIIEERPLTEMEIQAVNRKNQKWEREKLLECASISELLQPIINQIDSL |
| | FKDGNWYNDILPHVTYQDLKNIIIPELPKLKHWFIENLPGEYHEIE |
| | QKMKEALKYAFTQLDEKNLIHNGHFTTNLIDWQVEGDAQMKVL |
| | ENDALALQLFNWDASASQSINILEFDEDKAYKLRVYAQGSGTIQF |
| | GNCEDEAIQFNTNSFIYQEKIVYFDTPSVNLHIQSEGSEFIVSSIDLI |
| | ELSDDQ |

The cry14ab gene was synthesized and cloned it into a vector. The nucleic acid sequences encoding both the native Cry14Ab and the Cry14Ab with an altered N-terminal sequence are listed in Table 2.

The empty vector as well as the vector carrying cry14ab gene were introduced into the JBaCC expression system described in U.S. Pat. No. 11,484,568 the disclosure of which is incorporated herein in its entirety. The resultant bacterial strain expressing Cry14Ab was called Cry14Ab BaCC system.

TABLE 2
Nucleic acid sequence encoding Cry14Ab

| Sequence ID | cry 14ab nucleotide sequence |
|---|---|
| SEQ ID NO: 3 | atggattgtaatttacaatcacaacaaaatattccatataatgtattagcaataccagtatctaatgttaattcg |
| (encodes | ctttacaacaaggattttctgcttcacaaggaggaacattcaattatttaacattactacaatcaggaatatca |
| native | ttgactgatacagttggagatttaaaaaaagcatgggaagaatttcaaaaaactggttctttttcattaacag |
| Cry14Ab) | ttagctggttcttttgttcctggaggtacttttgtagcacctattattaatatggttattggttggttatggccaca |
| | taaaaacaaaaatgcggatacagaaaatttaataaatttaattgattcagaaattcaaaaacaattaaacaa |
| | agctttattagatgcagatagaaatgagtggagctcttatttagaatctatatttgattcttcaaataacctaaa |
| | tggtgcaattgtagatgcacagtggtcaggcactgtaaatactacaaatagaacactaagaaatccaaca |
| | gaatcagattatacaaatgttgttacaaattttattgcagcggatggtgacattgcaaataatgaaaatcaca |
| | taatgaatggcaactttgacgtagctgcagcaccttattttgttataggagcaacagcacgttttgcagcaa |
| | tgcaatcttatattaaattttgtaatgcttggattgataaagttggattgagtgacgcacagcttactacacaa |
| | aaggctaatttagatcgcacgaaacaaaatatgcgtaatgcaattcttaactatacacaacaagttatgaaa |
| | gtttttaaagattccaaaaatatgcctacaataggtactaataaatttagtgttgatacctataatgtatatatta |
| | aaggaatgacattaaatgttttagatattgtagcaatatggccttcattatatccagatgattatacttcacaa |
| | acagccttagaacaaacacgtgtcactttttcaaatatggttggccaagaagaaggtacagatggaagcc |
| | taagaatttacaatacttttgattcttttagttatcaacatagtccaatacctaataataatgttaatttaatttctta |
| | ttataatgatgaattacaaaatctagaattaggagtatatacccctcctaaaaaaggaagtggatactcttat |
| | ccttatggatttgttttaaattatgcaaacagtaaatataaatatggtgatagcaatgatccagaatctctagg |
| | aggattatctacactatctgcacctatacaacaagttaatgcagcaactcaaaacagtaaatatctagatgg |
| | agaaatcctaaatggaataggagcatccttacctggttattgtactacaggatgttcaccaacagaaccac |
| | cttttagttgtacttctaccgctaatggctataaagcaagctgtaatccttcagatacaaatcaaaaaattaac |
| | gctttatatccttttacacaagctaatgtaaagggaaacacaggaaaattaggagtactggcaagtcttgtt |
| | tcatatgatttaaatcctaaaaatgtatttggtgaattagattcagatacaaataatgttatcttaaaaggaatt |
| | cctgcagaaaaaggatattttcctaataatgcgcgtcctactgttgtaaaagaatggattaatggtgcaagt |
| | gctgtaccacttgattcaggaaataccttatttatgacggctacgaatttaacagctactcaatatagaatta |
| | gaatacgttatgcaaatccaaattcaaatactcaaatcggtgtacgaattacacaaaatggttctctaatttc |
| | cagtagtaatctaacactttatagtactactgatatgaataatactttaccactaaatgtatatgtaataggag |
| | aaaatggaaattatacacttcaagatttatataatactactaatgttttatcaacaggagatattacattacaaa |
| | ttacaggaggagatcaaaaaatatttattgatcgaatagaatttgttcctactatgcctgtacctggtaatact |
| | aacaacaataacggtaataataacggtaataataatcccccacaccacgtttgtgcaatagctggtacac |
| | aacaatcttgttctggaccgcccaaatttgaacaagtaagtgatttagaaaaaattacaacacaagtatata |
| | tgttattcaaatcttctccgtatgaagaattagctctagaagtttccagctatcaaattagtcaagtagcatta |
| | aaagttatggcattatctgatgaactattttgtgaagaaaaaaacgtattacgaaaattagtcaataaagca |
| | aaacaattattagaagcaagtaacttactagtaggtggaaattttgaaacaactcaaaattgggtacttgga |
| | acaaatgcttatataaattatgattcgtttttatttaatggaaattatttatctttacaaccagcaagtggatttttc |
| | acatcttatgcttatcaaaaaatagatgagtcaacattaaaaccatatacacgatataaagtttctgggttcat |
| | tgggcaaagtaatcaagtagaacttattatttctcgttatggaaaagaaattgataaaatattaaatgttccat |
| | atgcaggacctcttcctatcactgctgatgcatcaataacttgttgtgcaccagaaataggccaatgtgatg |
| | gggaacaatctgattctcatttctttaactatagcatcgatgtaggtgcacttcacccagaattaaaccctgg |
| | cattgaaattggtcttaaaattgtgcaatcaaatggttatataacaattagtaatctagaaattattgaagaac |
| | gtccacttacagaaatggaaattcaagcagtcaatcgaaaaaatcaaaaatgggaaagagaaaaacttc |
| | tagaatgtgcaagtattagtgaacttttacaaccaattattaatcaaatcgattcattgtttaaagatggaaac |
| | tggtataatgatattcttcctcatgtcacatatcaagatttaaaaaatattataatacccgagttaccaaaatta |
| | aaacattggttcatagagaatctcccaggtgaatatcatgaaattgaacaaaaaatgaaagaagctctaaa |
| | atatgcatttacacaattagacgagaaaaatttaatccacaatggtcactttacaactaacttaatagattgg |
| | caagtagaaggtgatgctcaaatgaaagtattagaaaatgatgctcttgcattacaacttttcaactgggat |
| | gctagtgcttcacaatctataaatatattagaatttgatgaagataaggcatataaacttcgcgtatatgctca |
| | aggaagcggaacaatccaatttggaaactgtgaagatgaagctatccaatttaatacaaactcattcatat |
| | atcaagaaaaaatagtctatttcgataccccatcagttaatttacacatacaatcagaaggttctgaatttatt |
| | gtaagtagtatcgatctaattgaattatcagacgaccaataa |
| SEQ ID NO: 4 | atgaatccgaacaatcgacaacaaaatattccatataatgtattagcaataccagtatctaatgttaattcgtt |
| (Encodes | gactgatacagttggagatttaaaaaaagcatgggaagaatttcaaaaaactggttctttttcattaacagct |
| Cry14Ab with | ttacaacaaggattttctgcttcacaaggaggaacattcaattatttaacattactacaatcaggaatatcatt |
| an altered N- | agctggttcttttgttcctggaggtacttttgtagcacctattattaatatggttattggttggttatggccacat |
| terminal | aaaaacaaaaatgcggatacagaaaatttaataaatttaattgattcagaaattcaaaaacaattaaacaaa |
| sequence) | gctttattagatgcagatagaaatgagtggagctcttatttagaatctatatttgattcttcaaataacctaaat |
| | ggtgcaattgtagatgcacagtggtcaggcactgtaaatactacaaatagaacactaagaaatccaaca |
| | gaatcagattatacaaatgttgttacaaattttattgcagcggatggtgacattgcaaataatgaaaatcaca |
| | taatgaatggcaactttgacgtagctgcagcaccttattttgttataggagcaacagcacgttttgcagcaa |
| | tgcaatcttatattaaattttgtaatgcttggattgataaagttggattgagtgacgcacagcttactacacaa |
| | aaggctaatttagatcgcacgaaacaaaatatgcgtaatgcaattcttaactatacacaacaagttatgaaa |
| | gtttttaaagattccaaaaatatgcctacaataggtactaataaatttagtgttgatacctataatgtatatatta |
| | aaggaatgacattaaatgttttagatattgtagcaatatggccttcattatatccagatgattatacttcacaa |
| | acagccttagaacaaacacgtgtcactttttcaaatatggttggccaagaagaaggtacagatggaagcc |
| | taagaatttacaatacttttgattcttttagttatcaacatagtccaatacctaataataatgttaatttaatttctta |
| | ttataatgatgaattacaaaatctagaattaggagtatatacccctcctaaaaaaggaagtggatactcttat |
| | ccttatggatttgttttaaattatgcaaacagtaaatataaatatggtgatagcaatgatccagaatctctagg |
| | aggattatctacactatctgcacctatacaacaagttaatgcagcaactcaaaacagtaaatatctagatgg |
| | agaaatcctaaatggaataggagcatccttacctggttattgtactacaggatgttcaccaacagaaccac |
| | cttttagttgtacttctaccgctaatggctataaagcaagctgtaatccttcagatacaaatcaaaaaattaac |
| | gctttatatccttttacacaagctaatgtaaagggaaacacaggaaaattaggagtactggcaagtcttgtt |
| | tcatatgatttaaatcctaaaaatgtatttggtgaattagattcagatacaaataatgttatcttaaaaggaatt |
| | cctgcagaaaaaggatattttcctaataatgcgcgtcctactgttgtaaaagaatggattaatggtgcaagt |
| | gctgtaccacttgattcaggaaataccttatttatgacggctacgaatttaacagctactcaatatagaatta |
| | gaatacgttatgcaaatccaaattcaaatactcaaatcggtgtacgaattacacaaaatggttctctaatttc |
| | cagtagtaatctaacactttatagtactactgatatgaataatactttaccactaaatgtatatgtaataggag |
| | aaaatggaaattatacacttcaagatttatataatactactaatgttttatcaacaggagatattacattacaaa |
| | ttacaggaggagatcaaaaaatatttattgatcgaatagaatttgttcctactatgcctgtacctggtaatact |
| | aacaacaataacggtaataataacggtaataataatcccccacaccacgtttgtgcaatagctggtacac |
| | aacaatcttgttctggaccgcccaaatttgaacaagtaagtgatttagaaaaaattacaacacaagtatata |
| | tgttattcaaatcttctccgtatgaagaattagctctagaagtttccagctatcaaattagtcaagtagcatta |
| | aaagttatggcattatctgatgaactattttgtgaagaaaaaaacgtattacgaaaattagtcaataaagca |
| | aaacaattattagaagcaagtaacttactagtaggtggaaattttgaaacaactcaaaattgggtacttgga |
| | acaaatgcttatataaattatgattcgtttttatttaatggaaattatttatctttacaaccagcaagtggatttttc |
| | acatcttatgcttatcaaaaaatagatgagtcaacattaaaaccatatacacgatataaagtttctgggttcat |
| | tgggcaaagtaatcaagtagaacttattatttctcgttatggaaaagaaattgataaaatattaaatgttccat |
| | atgcaggacctcttcctatcactgctgatgcatcaataacttgttgtgcaccagaaataggccaatgtgatg |
| | gggaacaatctgattctcatttctttaactatagcatcgatgtaggtgcacttcacccagaattaaaccctgg |
| | cattgaaattggtcttaaaattgtgcaatcaaatggttatataacaattagtaatctagaaattattgaagaac |
| | gtccacttacagaaatggaaattcaagcagtcaatcgaaaaaatcaaaaatgggaaagagaaaaacttc |
| | tagaatgtgcaagtattagtgaacttttacaaccaattattaatcaaatcgattcattgtttaaagatggaaac |
| | tggtataatgatattcttcctcatgtcacatatcaagatttaaaaaatattataatacccgagttaccaaaatta |
| | aaacattggttcatagagaatctcccaggtgaatatcatgaaattgaacaaaaaatgaaagaagctctaaa |
| | atatgcatttacacaattagacgagaaaaatttaatccacaatggtcactttacaactaacttaatagattgg |
| | caagtagaaggtgatgctcaaatgaaagtattagaaaatgatgctcttgcattacaacttttcaactgggat |
| | gctagtgcttcacaatctataaatatattagaatttgatgaagataaggcatataaacttcgcgtatatgctca |
| | aggaagcggaacaatccaatttggaaactgtgaagatgaagctatccaatttaatacaaactcattcatat |
| | atcaagaaaaaatagtctatttcgataccccatcagttaatttacacatacaatcagaaggttctgaatttatt |
| | gtaagtagtatcgatctaattgaattatcagacgaccaataa |

### Example 9: Inhibition of Larval Development of H. contortus and Cyathostomins by Cry14Ab

Efficacy of Cry14Ab to inhibit larval development from eggs was tested using the methods described by Hu, Y., et al. PLoS NEGL TROP DIs 4, e6506 (2018). Larval development inhibition assays were set up in 96-well plates using 60 eggs of H. contortus or eggs of Cyathostomins L3i in each well. Varying amounts 0.01 ng/ml to 1100 ng/ml of a bacterial strain KFBT carrying either an empty vector (KFBT-43 (EV)) or a vector expressing Cry14Ab (KFBT-72 (Cry14Ab)) were added to the wells. Plates were incubated at 28° C. for 7 days. Number of larval in each well measured.

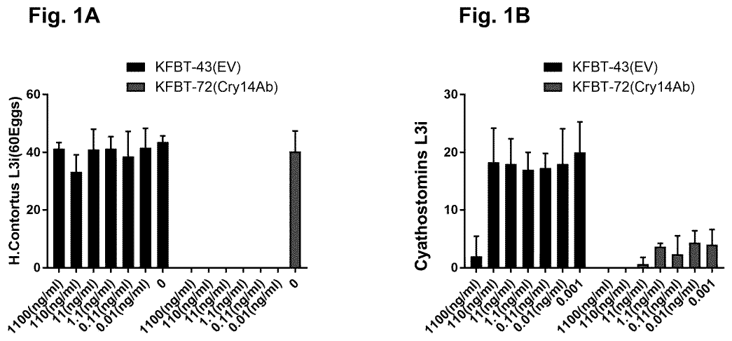

Bacteria expressing Cry14Ab completely inhibited hatching of eggs and larval development at all concentrations of bacteria tested for both H. contortus (FIG. 1A) and Cyathostomins L3i (FIG. 1B).

### Example 10: In Vivo Efficacy of Cry14Ab for Reduction of Fecal Egg Counts and Worm Burdens of H. contortus in Sheep

To test the efficacy of Cry14Ab for reducing fecal eggs counts and worm burdens of 20 H. contortus in sheep methods described by Sanders et al., IJPDDR 14(2020): 30-236 were modified to test a single dose of Cry14Ab. Dorset lambs were infected with 10,000 HC infective larvae. Once the infection was patent, lambs were stratified by fecal egg count (FEC) and sequentially assigned into one of three treatment groups (n=5). Lambs were orally administered either a single dose of CryAb IBaCC lysate at 30 or 15 mg/kg BW or were untreated (received a dose of water). FEC were measured daily until lambs were euthanized at day seven and total worm burdens were quantified and sexed from the recovered abomasums.

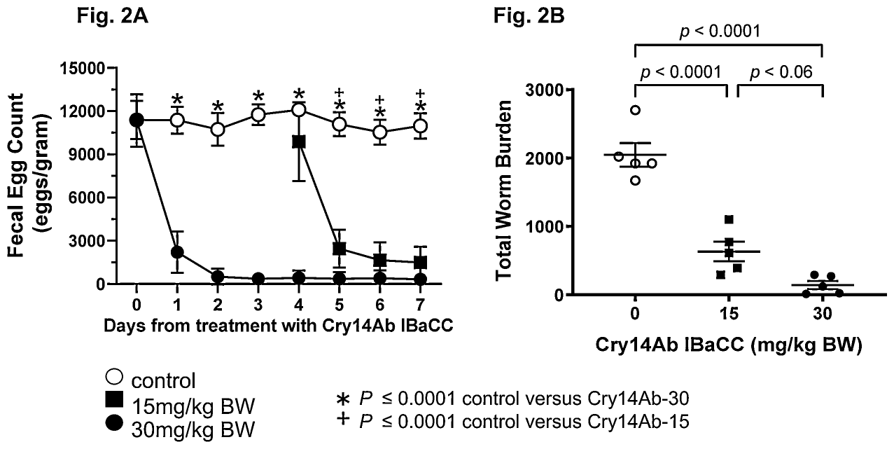

FIGS. 2A-2B show that compared to the controls with empty vector IBaCC, the Cry14Ab IBaCC lysate significantly reduced both fecal egg counts (FIG. 2A) and worm burdens (FIG. 2B) of H. contortus in sheep.

### Example 11: In Vitro Bioactivity of Cry14Ab Against Ascaris suum Larvae

Bioactivity of Cry14Ab against A. suum was tested in vitro using methods described by Urban et al (Urban et al., PloS NTD 7(6): e2263 (2013)). A. suum L4s larvae were added to wells of a 24-well plate. Cry14Ab IbaCC lysate was added to test wells at a concentration of 40 μg/ml. 20 mM Hepes pH 8.0 buffer was added in place of Cry14Ab to control wells. The assays were scored daily over the period of one week. Larvae that moved in the absence or presence of gentle touch with an eyelash were scored as alive; larvae that did not move were scored as dead.

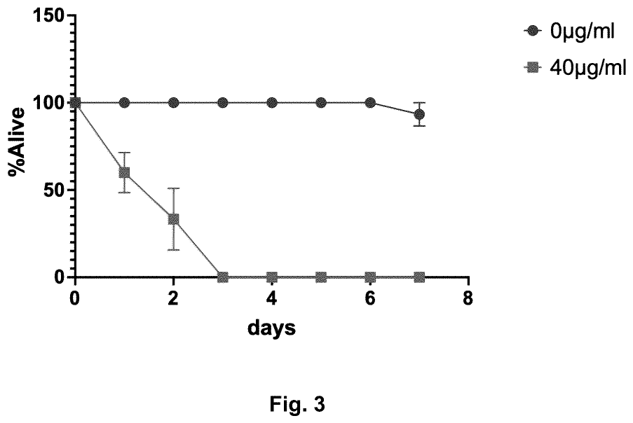

FIG. 3 shows that Cry14Ab IbaCC lysate significantly reduced viability of A. suum larvae in the first 2 days and killed all larvae by day 3.

### Example 12: In Vitro Bioactivity of Cry14Ab Against Whipworm Trichuris muris

Bioactivity of Cry14Ab against T. muris adult worms was tested in vitro using methods described by Hu et al. (PLOS ONE 8(7) (2013)). T. muris in vitro assays were carried out in a 2.0 mL volume in 12-well plates using five adults per well (separated by gender) harvested between days 35-40 P. I. Plates were put at 37° C. in a 5% CO2 in air incubator. Cry14Ab IBaCC lysate (at a dose ranging from 0 (control) to 1000 μg/ml was added to the wells). Motility of each parasitic nematode in each well was scored. The motility of each adult was assessed as either +(moved with or without touching) or—(did not move even when touched). Motility data was averaged by combining all the data from all replicates and treating them as one experiment.

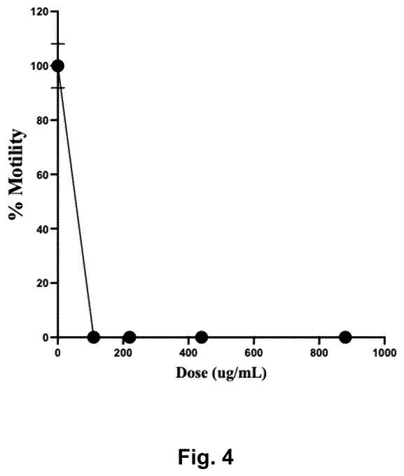

FIG. 4 shows that even at a low dose of about 100 μg/mL, Cry14Ab IBaCC lysate significantly reduced motility of adults T. muris worms in vitro.

### Example 13: In Vivo Efficacy of Cry14Ab for Reduction of Fecal Egg Counts and Worm Burdens of Ancylostoma Celyanicum in Hamsters

In vivo efficacy of Cry14Ab IBaCC lysate for reducing fecal eggs counts and worm burdens of A. celyanicum in hamsters was tested using methods described by Hu, Y., et al. PLoS NEGL TROP DIs 4, e6506 (2018).

Male hamsters were infected with A. celyanicum and on day 17 post-inoculation (P.I.), an overnight fecal sample was collected from each infected hamster. The number of eggs present was counted using the modified McMaster technique and the hamsters were grouped to ensure that the hamsters in each treatment group had roughly equivalent infection levels. On day 18 P.I., hamsters were individually weighed and given a single dose of 20 mg per kg of body weight of Cry14Ab IBaCC lysate or water (control) through a blunt-ended gavage needle. On day 21 P.I., an overnight fecal sample was collected from each infected hamster. The hamsters were sacrificed on day 22 P.I., and parasite burdens and eggs per gram of feces were determined.

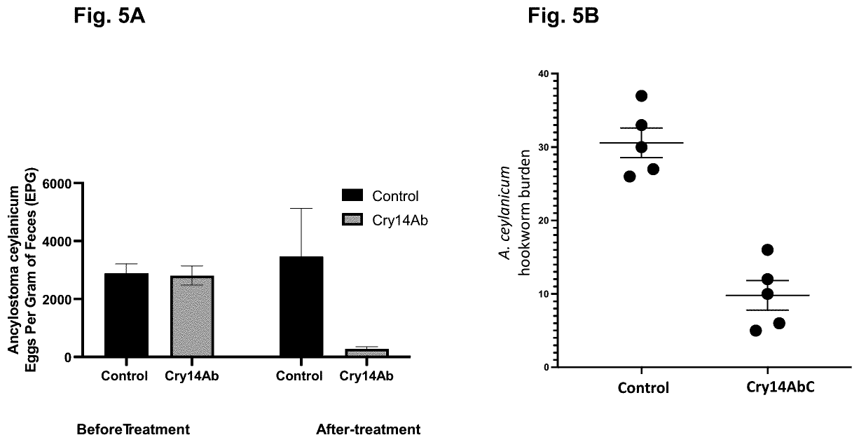

FIGS. 5A-5B show that treatment with a single dose of Cry14Ab IBaCC lysate significantly reduced the fecal egg counts (FIG. 5A) as well as worm burdens (FIG. 5B) of A. celyanicum in infected hamsters.

### Example 14: In Vivo Efficacy of Cry14Ab for Reduction of Fecal Egg Counts and Worm Burdens of Heligmosomoides Bakeri in Mice

In vivo efficacy of Cry14Ab IBaCC lysate for reducing fecal eggs counts and worm burdens of A. celyanicum in mice was tested using methods described by Hu et al. (PLOS ONE 8(7) (2013)).

On day 0, mice were infected per os with a suspension of 200610 H. bakeri L3 larvae in 0.1 mL of distilled water. Larvae were counted under the microscope, then drawn into a pipette tip and placed into separate glass test tubes until gavage with a blunt ended syringe. On days 14, 16, 18, and 20 post-infection (P.I.), fecal samples were collected from the mice. Mice were placed individually in empty plastic cages for 1 h each morning, and the fecal pellets were collected into 50 mL centrifuge tubes. The number of eggs present was counted using the modified McMaster technique. Briefly, feces collected from mice were weighed and resuspended in a 1 g:15 mL volume of water. The pellets were allowed to soak overnight before being broken up for 1 h via heavy vortexing. The eggs were counted using a 2-chamber McMaster slide, each chamber holding a 0.6 mL volume of a 1:1 mixture of fecal slurry and saturated sucrose solution. The number of eggs per gram of feces was thus calculated from the following equation: number of eggs counted× (1/0.3 mL slurry)×(15 mL slurry/g feces). For each mouse and each time point, three different egg counts were made and then averaged. Each mouse was treated per os on day 15 P.I. with a single dose of placebo or 50 mg of Cry14Ab IBaCC lysate per kg body weight through a blunt-ended syringe. All mice were killed by exposure to CO2 on day 20 P.I. and the intestines were removed in their entirety. These were opened longitudinally with a pair of blunt-ended dissecting scissors and then placed into a 50 mL centrifuge tube with 10-20 mL of pre-warmed (37° C.) PBS for approximately 1 h to allow worms to dislodge from the intestine. The solution and intestine were examined under a microscope, using fine tweezers when necessary for further extrication of worms from the intestine, for determination of final worm burden.

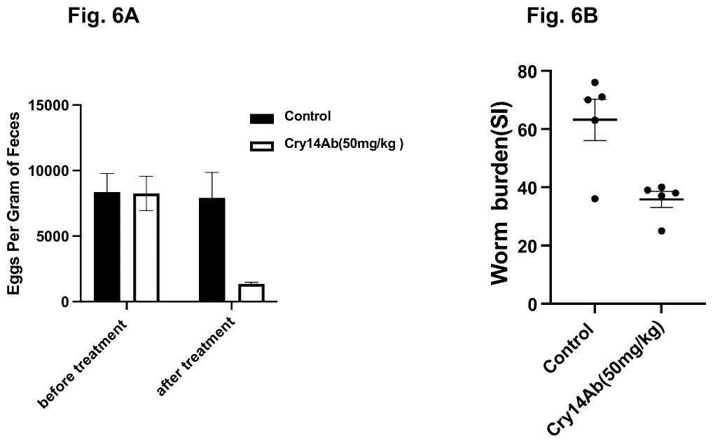

FIGS. 6A-6B show that treatment with a single 50 mg dose of Cry14Ab IBaCC lysate significantly reduced the fecal egg counts (FIG. 6A) as well as worm burdens (FIG. 6B) of H. bakeri in infected mice.

### Example 15: In Vivo Efficacy of Cry14Ab for Reduction of Fecal Egg Counts and Worm Burdens of Necator americanus Hookworm in Hamsters

In vivo efficacy of Cry14Ab IBaCC lysate for reducing fecal eggs counts and worm burdens of N. americanus in hamsters was tested using methods described by Hu et al. (PLOS ONE 8(7) (2013)).

On day 0, hamsters (N=3 per group) were infected per os with a suspension of 200610 N. americanus L3 larvae in 0.1 mL of distilled water. Larvae were counted under the microscope, then drawn into a pipette tip and placed into separate glass test tubes until gavage with a blunt ended syringe. Hamsters were treated with a single oral dose of water (control) or 50 mg/kg Cry14Ab IBaCC lysate. On days 14, 16, 18, and 20 post-infection (P.I.), fecal samples were collected from the hamsters. Hamsters were placed individually in empty plastic cages for 1 h each morning, and the fecal pellets were collected into 50 mL centrifuge tubes. The number of eggs present was counted using the modified McMaster technique. Briefly, feces collected from hamsters were weighed and resuspended in a 1 g:15 mL volume of water. The pellets were allowed to soak overnight before being broken up for 1 h via heavy vortexing. The eggs were counted using a 2-chamber McMaster slide, each chamber holding a 0.6 mL volume of a 1:1 mixture of fecal slurry and saturated sucrose solution. The number of eggs per gram of feces was thus calculated from the following equation: number of eggs counted×(1/0.3 mL slurry)×(15 mL slurry/g feces). For each hamster and each time point, three different egg counts were made and then averaged. Each hamster was treated per os on day 15 P.I. with a single dose of placebo or 50 mg of Cry14Ab IBaCC lysate per kg body weight through a blunt-ended syringe. All hamsters were killed by exposure to CO2 on day 20 P.I. and the intestines were removed in their entirety. These were opened longitudinally with a pair of blunt-ended dissecting scissors and then placed into a 50 mL centrifuge tube with 10-20 mL of pre-warmed (37° C.) PBS for approximately 1 h to allow worms to dislodge from the intestine. The solution and intestine were examined under a microscope, using fine tweezers when necessary for further extrication of worms from the intestine, for determination of final worm burden.

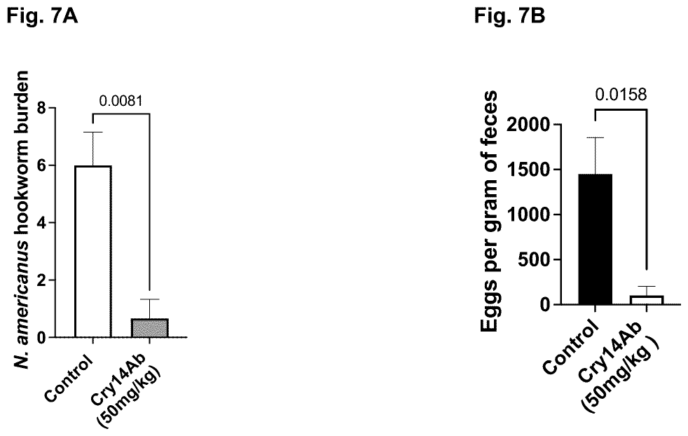

FIGS. 7A-7B show that treatment with a single 50 mg oral dose of Cry14Ab IBaCC lysate significantly reduced the worm burdens (FIG. 7A) and fecal egg counts (FIG. 7B) of N. americanus in infected hamsters.

### Example 16: In Vitro and In Vivo Bioactivity of Cry14Ab Against Roundworm Ascaris suum

Bioactivity of Cry14Ab was tested against intestinal L4 staged A. suum (pig roundworm) in vitro using methods described by Hu et al. (PLOS ONE 8(7) (2013)). Fourth staged (L4) parasite A. suum larvae were harvested from the intestines of infected mice and studied their motility in 48-well format over time using the touch 3-0 scale (3=fully motile; 2=inhibited motility; 1=immotile until touched; 0=immotile even with touch). A. suum in vitro assays were carried out in a 2.0 mL volume in 12-well plates using four or five adults per well (separated by gender) harvested between days 35-40 P.I. Plates were put at 37° C. in a 5% CO2 in air incubator. Cry14Ab IBaCC lysate (at a dose of 0 (control), 50 μg/ml, and 100 μg/ml was added to the wells). Motility of each parasitic nematode in each well was scored. The motility of each adult was assessed and motility data was averaged by combining all the data from all replicates and treating them as one experiment (n=20 per dose; 5 per experiments repeated 4 times).

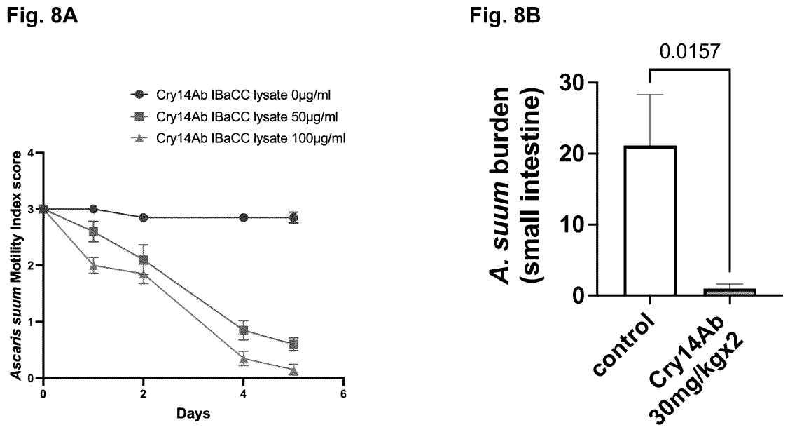

FIG. 8A shows that both the 50 and 100 μg/mL doses of Cry14Ab IBaCC lysate significantly reduced motility of adults A. suum worms in vitro.

In vivo efficacy of Cry14Ab IBaCC lysate for reducing worm burdens of T. suum in mice was tested using methods described by Hu et al. (PLOS ONE 8(7) (2013)).

On day 0, mice were infected per os with a suspension of A. suum L3 larvae in 0.1 mL of distilled water. Larvae were counted under the microscope, then drawn into a pipette tip and placed into separate glass test tubes until gavage with a blunt ended syringe. A. suum were allowed to progress to the intestinal phase. Mice were treated with a double oral dose of Cry14Ab or water (control). All mice were killed by exposure to CO₂ on day 20 P.I. and the intestines were removed in their entirety. These were opened longitudinally with a pair of blunt-ended dissecting scissors and then placed into a 50 mL centrifuge tube with 10-20 mL of pre-warmed (37° C.) PBS for approximately 1 h to allow worms to dislodge from the intestine. The solution and intestine were examined under a microscope, using fine tweezers when necessary for further extrication of worms from the intestine, for determination of final worm burden.

FIG. 8B shows that treatment with a double dose of 30 mg of Cry14Ab IBaCC lysate significantly reduced the worm burdens of T. suum in infected mice.

### REFERENCES

1. Bethony J, Brooker S, Albonico M, Geiger S M, Loukas A, Diemert D, Hotez P J. 2006. Soil-transmitted helminth infections: ascariasis, trichuriasis, and hookworm. Lancet 367:1521-1532.
2. Bowman D D (1999) Georgis' Parasitology for Veterinarians 7th Edition ed: W.B. Saunders Company. pp 290-291.
3. Hall A, Hewitt G, Tuffrey V, de Silva N. 2008. A review and meta analysis of the impact of intestinal worms on child growth and nutrition. Matern. Child Nutr. 4 (Suppl 1):118-236.
4. Knopp S, Steinmann P, Keiser J, Utzinger J. 2012. Nematode infections: soil-transmitted helminths and trichinella. Infect. Dis. Clin. North Am. 26:341-358.
5. Tchuem Tchuente L A. 2011. Control of soil-transmitted helminths in sub-Saharan Africa: diagnosis, drug efficacy concerns and challenges. Acta Trop. 120 (Suppl 1):S4-S11.
6. Hotez P J. 2008. Forgotten people, forgotten diseases: the neglected tropical diseases and their impact on global health and development. ASM Press, Washington, D C.
7. Keiser J, Utzinger J. 2010. The drugs we have and the drugs we need against major helminth infections. Adv. Parasitol. 73:197-230.
8. Humphries D, Mosites E, Otchere J, Twum W A, Woo L, Jones-Sanpei H, Harrison L M, Bungiro R D, Benham-Pyle B, Bimi L, Edoh D, Bosompem K, Wilson M, Cappello M. 2011. Epidemiology of hookworm infection in Kintampo North Municipality, Ghana: patterns of malaria coinfection, anemia, and albendazole treatment failure. Am. J. Trop. Med. Hyg. 84:792-800.
9. Soukhathammavong P A, Sayasone S, Phongluxa K, Xayaseng V, Utzinger J, Vounatsou P, Hatz C, Akkhavong K, Keiser J, Odermatt P. 2012. Low efficacy of single-dose albendazole and mebendazole against hookworm and effect on concomitant helminth infection in Lao PDR. PLoS Negl. Trop. Dis. 6:e1417. doi:10.1371/journal.pntd.0001417.
10. Stothard J R, Rollinson D, Imison E, Khamis I S. 2009. A spot-check of the efficacies of albendazole or levamisole, against soil-transmitted helminthiases in young Ungujan children, reveals low frequencies of cure. Ann. Trop. Med. Parasitol. 103:357-360.
11. Geary T G, Woo K, McCarthy J S, Mackenzie C D, Horton J, Prichard R K, de Silva N R, Olliaro P L, Lazdins-Helds J K, Engels D A, Bundy D A. 2010. Unresolved issues in anthelmintic pharmacology for helminthiases of humans. Int. J. Parasitol. 40:1-13.
12. Holden-Dye L, Walker R J. 2007. Anthelmintic drugs. WormBook 2007: 1-13.
13. Cappello M, Bungiro R D, Harrison L M, Bischof L J, Griffitts J S, Barrows B D, Aroian R V. 2006. A purified Bacillus thuringiensis crystal protein with therapeutic activity against the hookworm parasite Ancylostoma ceylanicum. Proc. Natl. Acad. Sci. U.S.A 103:15154-15159.
14. Hu Y, Ellis B L, Yiu Y Y, Miller M M, Urban J F, et al. (2013) An Extensive Comparison of the Effect of Anthelmintic Classes on Diverse Nematodes. PLOS ONE 8(7): e70702. https://doi.org/10.1371/journal.pone.0070702
15. Hu Y, Georghiou S B, Kelleher A J, Aroian R V. 2010. Bacillus thuringiensis Cry5B protein is highly efficacious as a single-dose therapy against an intestinal roundworm infection in mice. PLoS Negl. Trop. Dis. 4:e614. doi:10.1371/journal.pntd.0000614.
16. Hu Y, Zhan B, Keegan B, Yiu Y Y, Miller M M, Jones K, Aroian R V. 2012. Mechanistic and single-dose in vivo therapeutic studies of Cry5B anthelmintic action against hookworms. PLoS Negl. Trop. Dis. 6:e1900. doi:10.1371/journal.pntd.0001900.
17. Cutting S M. 2011. Bacillus probiotics. Food Microbiol. 28:214-220.
18. Casula G, Cutting S M. 2002. Bacillus probiotics: spore germination in the gastrointestinal tract. Appl. Environ. Microbiol. 68:2344-2352.
19. Duc L H, Hong H A, Barbosa T M, Henriques A O, Cutting S M. 2004. Characterization of Bacillus probiotics available for human use. Appl. Environ. Microbiol. 70:2161-2171.
20. Hoa N T, Baccigalupi L, Huxham A, Smertenko A, Van P H, Ammendola S, Ricca E, Cutting A S. 2000. Characterization of Bacillus species used for oral bacteriotherapy and bacterioprophylaxis of gastrointestinal disorders. Appl. Environ. Microbiol. 66:5241-5247.
21. Hoa T T, Duc L H, Isticato R, Baccigalupi L, Ricca E, Van P H, Cutting S M. 2001. Fate and dissemination of Bacillus subtilis spores in a murine model. Appl. Environ. Microbiol. 67:3819-3823.
22. Hong H A, Huang J M, Khaneja R, Hiep L V, Urdaci M C, Cutting S M. 2008. The safety of Bacillus subtilis and Bacillus indicus as food probiotics. J. Appl. Microbiol. 105:510-520.
23. D'Arienzo R, Maurano F, Mazzarella G, Luongo D, Stefanile R, Ricca E, Rossi M. 2006. Bacillus subtilis spores reduce susceptibility to Citrobacter rodentium-mediated enteropathy in a mouse model. Res. Microbiol. 157: 891-897.
24. Duc L H, Hong H A, Fairweather N, Ricca E, Cutting S M. 2003. Bacterial spores as vaccine vehicles. Infect. Immun. 71:2810-2818.
25. Hoang T H, Hong H A, Clark G C, Titball R W, Cutting S M. 2008. Recombinant Bacillus subtilis expressing the Clostridium perfringens alpha toxoid is a candidate orally delivered vaccine against necrotic enteritis. Infect. Immun. 76:5257-5265.
26. La Ragione R M, Casula G, Cutting S M, Woodward M J. 2001. Bacillus subtilis spores competitively exclude Escherichia coli 078:K80 in poultry. Vet. Microbiol. 79:133-142.
27. La Ragione R M, Woodward M J. 2003. Competitive exclusion by Bacillus subtilis spores of Salmonella enterica serotype Enteritidis and Clostridium perfringens in young chickens. Vet. Microbiol. 94:245-256.
28. Permpoonpattana P, Hong H A, Phetcharaburanin J, Huang J M, Cook J, Fairweather N F, Cutting S M. 2011. Immunization with Bacillus spores expressing toxin A peptide repeats protects against infection with Clostridium difficile strains producing toxins A and B. Infect. Immun. 79: 2295-2302.
29. Song M, Hong H A, Huang J M, Colenutt C, Khang D D, Nguyen T V, Park S M, Shim B S, Song H H, Cheon I S, Jang J E, Choi J A, Choi Y K, Stadler K, Cutting S M. 2012. Killed Bacillus subtilis spores as a mucosal adjuvant for an H5N1 vaccine. Vaccine 30:3266-3277.
30. Conlan J V, Khamlome B, Vongxay K, Elliot A, Pallant L, Sripa B, Blacksell S D, Fenwick S, Thompson R C. 2012. Soil-transmitted helminthiasis in Laos: a community-wide cross-sectional study of humans and dogs in a mass drug administration environment. Am. J. Trop. Med. Hyg. 86:624-634.
31. Marroquin L D, Elyassnia D, Griffitts J S, Feitelson J S, Aroian R V. 2000. Bacillus thuringiensis (Bt) toxin susceptibility and isolation of resistance mutants in the nematode Caenorhabditis elegans. Genetics 155:1693-1699.
32. Dubnau D, Davidoff-Abelson R. 1971. Fate of transforming DNA following uptake by competent Bacillus subtilis. I. Formation and properties of the donor-recipient complex. J. Mol. Biol. 56:209-221.
33. Sierro N, Makita Y, de Hoon M, Nakai K. 2008. DBTBS: a database of transcriptional regulation in Bacillus subtilis containing upstream intergenic conservation information. Nucleic Acids Res. 36:D93-D96.
34. Shevchenko A, Wilm M, Vorm O, Mann M. 1996. Mass spectrometric sequencing of proteins silver-stained polyacrylamide gels. Anal. Chem. 68:850-858.
35. National Research Council. 1996. Guide for the care and use of laboratory animals. National Academies Press, Washington, D C.
36. Hu Y, Xiao S H, Aroian R V. 2009. The new anthelmintic tribendimidine is an L-type (levamisole and pyrantel) nicotinic acetylcholine receptor agonist. PLoS Negl. Trop. Dis. 3:e499. doi:10.1371/journal.pntd.0000499.
37. Hu Y, Platzer E G, Bellier A, Aroian R V. 2010. Discovery of a highly synergistic anthelmintic combination that shows mutual hypersusceptibility. Proc. Natl. Acad. Sci. U.S.A. 107:5955-5960.
38. Lereclus D, Arantes O, Chaufaux J, Lecadet M. 1989. Transformation and expression of a cloned delta-endotoxin gene in Bacillus thuringiensis. FEMS Microbiol. Lett. 51:211-217.
39. Yang Y, Qi Y, Huang Y. 1996. Cloning and expression of full-length delta-endotoxin crylA(c) gene in three kinds of prokaryotic systems using shuttle vector pHT3101. Wei Sheng Wu Xue Bao 36:173-180.
40. Youngman P, Perkins J B, Losick R. 1984. Construction of a cloning site near one end of Tn917 into which foreign DNA may be inserted without affecting transposition in Bacillus subtilis or expression of the transposonborne erm gene. Plasmid 12:1-9.
41. Cannon R J C. 1996. Bacillus thuringiensis use in agriculture: a molecular perspective. Biol. Rep. 71:561-636.
42. Hu Y, Aroian R V. 2012. Promise of Bacillus thuringiensis crystal proteins as anthelmintics, p 267-281. In Caffrey C R (ed), Parasitic helminths: targets, screens, drugs, and vaccines. Wiley-VCH Verlag Gmh & Co, KGaA, Weinheim, Germany.
43. Bischof L J, Huffman D L, Aroian R V. 2006. Assays for toxicity studies in C. elegans with Bt crystal proteins. Methods Mol. Biol. 351:139-154.
44. Kho M F, Bellier A, Balasubramani V, Hu Y, Hsu W, Nielsen-LeRoux C, McGillivray S M, Nizet V, Aroian R V. 2011. The pore-forming protein Cry5B elicits the pathogenicity of Bacillus sp. against Caenorhabditis elegans. PLoS One 6:e29122. doi:10.1371/journal.pone.0029122.
45. Baum J A, Malvar T. 1995. Regulation of insecticidal crystal protein production in Bacillus thuringiensis. Mol. Microbiol. 18:1-12.
46. Buasri W, Panbangred W. 2012. Large crystal toxin formation in chromosomally engineered Bacillus thuringiensis subsp. aizawai due to sigmaE accumulation. Appl. Environ. Microbiol. 78:1682-1691.
47. Brans A, Filee P, Chevigne A, Claessens A, Joris B. 2004. New integrative method to generate Bacillus subtilis recombinant strains free of selection markers. Appl. Environ. Microbiol. 70:7241-7250.
48. Tritten L, Nwosu U, Vargas M, Keiser J. 2012. In vitro and in vivo efficacy of tribendimidine and its metabolites alone and in combination against the hookworms Heligmosomoides bakeri and Ancylostoma ceylanicum. Acta Trop. 122:101-107.
49. Tritten L, Silbereisen A, Keiser J. 2011. In vitro and in vivo efficacy of monepantel (AAD 1566) against laboratory models of human intestinal nematode infections. PLoS Negl. Trop. Dis. 5:e1457. doi:10.1371/journal.pntd.0001457.
50. Griffitts J S, Aroian R V. 2005. Many roads to resistance: how invertebrates adapt to Bt toxins. Bioessays 27:614-624.
51. Griffitts J S, Haslam S M, Yang T, Garczynski S F, Mulloy B, Morris H, Cremer P S, Dell A, Adang M J, Aroian R V. 2005. Glycolipids as receptors for Bacillus thuringiensis crystal toxin. Science 307:922-925.
52. Los F C, Kao C Y, Smitham J, McDonald K L, Ha C, Peixoto C A, Aroian R V. 2011. RAB-5- and RAB-11-dependent vesicle-trafficking pathways are required for plasma membrane repair after attack by bacterial poreforming toxin. Cell Host Microbe 9:147-157.
53. Wang F, Liu Y, Zhang F, Chai L, Ruan L, Peng D, Sun M. 2012. Improvement of crystal solubility and increasing toxicity against Caenorhabditis elegans by asparagine substitution in block 3 of Bacillus thuringiensis crystal protein Cry5Ba. Appl. Environ. Microbiol. 78:7197-7204.
54. el-Bendary M A. 2006. Bacillus thuringiensis and Bacillus sphaericus biopesticides production. J. Basic Microbiol. 46:158-170.
55. Schallmey M, Singh A, Ward O P. 2004. Developments in the use of Bacillus species for industrial production. Can. J. Microbiol. 50:1-17.
56. Fujiwara R T, Geiger S M, Bethony J, Mendez S. 2006. Comparative immunology of human and animal models of hookworm infection. Parasite Immunol.
57. Stepek G, Lowe A E, Buttle D J, Duce I R, Behnke J M. 2007. Anthelmintic action of plant cysteine proteinases against the rodent stomach nematode, Protospirura muricola, in vitro and in vivo. Parasitology.
58. Hu Y, Ellis B L, Yiu Y Y, Miller M M, Urban J F, Shi L Z, Aroian R V. 2013. An extensive comparison of the effect of anthelmintic classes on diverse nematodes. PLoS One.
59. Lee A C, Epe C, Simpson K W, Bowman D D. 2011. Utility of capsule endoscopy for evaluating anthelmintic efficacy in fully conscious dogs. Int. J. Parasitol.
60. Lee A C, Epe C, Bowman D D. 2015. Determination of anthelmintic efficacy against Toxocara canis in dogs by use of capsule endoscopy. Vet. Parasitol.
61. Agaisse H, Lereclus D. 1994. Structural and functional analysis of the promoter region involved in full expression of the cryIIIA toxin gene of Bacillus thuringiensis. Mol. Microbiol.
62. Boontawan A, Stuckey D C. 2005. Mass transfer of terpenes through a silicone rubber membrane in a liquid-liquid contacting system. Biotechnol. Prog.
63. Krings U, Berger R G. 1998. Biotechnological production of Oavours and fragrances. Appl. Microbiol. Biotechnol.
64. Chan A C, Ager D, Thompson I P. 2013. Resolving the mechanism of bacterial inhibition by plant secondary metabolites employing a combination of whole-cell biosensor. J. Microbiol. Methods.
65. Lereclus D, Agaisse H, Gominet M, Chaufaux J. 1995. Overproduction of Encapsulated Insecticidal Crystal Proteins in a Bacillus thuringiensis spo0A Mutant. Nat. Biotechnol.
66. Agaisse H, Lereclus D. 1994. Expression in Bacillus subtilis of the Bacillus thuringiensis cryIIIA toxin gene is not dependent on a sporulation-specific sigma factor and is increased in a spo0A mutant. J. Bacteriol.
67. Urban Jr J F, Hu Y, Miller M M, Scheib U, Yiu Y Y, Aroian R V. 2013. Bacillus thuringiensis-derived Cry5B has potent anthelmintic activity against Ascaris suum. PLoS Negl. Trop. Dis.
68. Roh J Y, Choi J Y, Li M S, Jin B R, Je Y H. 2007. Bacillus thuringiensis as a specific, safe, and effective tool for insect pest control. J. Microbiol. Biotechnol.
69. Hu Y, Miller M M, Derman A I, Ellis B L, Monnerat R G, Pogliano J, Aroian R V. 2013. Bacillus subtilis strain engineered for treatment of soil-transmitted helminth diseases. Appl. Environ. Microbiol.
70. Silvaggi, J., et al. Unmasking novel sporulation genes in Bacillus subtillus. J Bacteriol. 186, 8089-8095, 2004.
71. Sandman, K., et al. Genetic Analysis of Bacillus subtilis spo Mutations Generated by Tn917-Mediated Insertional Mutagenesis. Genetics. 117, 603-617, 1987.
72. Malvar and Baum, Tn5401 Disruption of the spo0F Gene, Identified by Direct Chromosomal Sequencing, Results in CrylIlIA Overproduction in Bacillus thuringiensis. J Bacteriol. 176, 4750-4753, 1994.
73. Sanders J, et al. A new paraprobiotic-based treatment for control of Haemonchus contortus in sheep. Int J Parasitol Drugs Drug Resist. 2020 December; 14:230-236. doi: 10.1016/j.ijpddr.2020.11.004. Epub 2020 Nov. 19. PMID: 33242790; PMCID: PMC7695930.
74. Hu Y, Miller M, Zhang B, Nguyen T T, Nielsen M K, et al. (2018) In vivo and in vitro studies of Cry5B and nicotinic acetylcholine receptor agonist anthelmintics reveal a powerful and unique combination therapy against intestinal nematode parasites. PLOS Neglected Tropical Diseases 12(5): e0006506. Website: doi.org/10.1371/journal.pntd.0006506
75. Li H, Abraham A, Gazzola D, Hu Y, Beamer G, Flanagan K, Soto E, Rus F, Mirza Z, Draper A, Vakalapudi S, Stockman C, Bain P, Urban J F Jr, Ostroff G R, Aroian R V. Antimicrob Agents Chemother. 2021 Feb. 17; 65 (3):e01469-20. doi: 10.1128/AAC.01469-20. Print 2021 Feb. 17.
76. Kahn T W, Duck N B, McCarville M T, Schouten L C, Schweri K, Zaitseva J, Daum J. A Bacillus thuringiensis Cry protein controls soybean cyst nematode in transgenic soybean plants. Nat Commun. 2021 Jun. 7; 12(1):3380. doi: 10.1038/s41467-021-23743-3. PMID: 34099714; PMCID: PMC8184815.

All publications and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

While particular steps, elements, embodiments and applications of the present invention have been shown and described herein for purposes of illustration, it will be understood, of course, that the invention is not limited thereto since modifications may be made by persons skilled in the art, particularly in light of the foregoing teachings, without deviating from the spirit and scope of the invention. Accordingly, the invention is not limited except as by the appended claims.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications, and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A composition comprising a killed or inactivated non-sporulating bacterium that is genetically engineered to express Cry14Ab in the cytosol of the bacterium.

2. The composition of claim 1, wherein:
the killed or inactivated bacterium is genetically engineered to have a genetic mutation that results in a defect in sporulation such that Cry14Ab is trapped in the cytosol of the bacterium; or
the expression of the gene encoding Cry14Ab is under control of a non-sporulation-specific promoter, optionally wherein the promoter is a Cry3A, GerA, GNAT, or TadA promoter.

3-4. (canceled)

5. The composition of claim 1, wherein the bacterium is:
(a) a Gram-positive bacterium,
(b) a Gram-negative bacterium;
(c) a species of Bacillus;
(d) Bacillus thuringiensis (Bt);
(e) E. coli; or
(f) P. fluorescens.

6-7. (canceled)

8. The composition of claim 1, wherein the bacterium has a genetic mutation, wherein the genetic mutation is a deletion or inactivation of one or more genes resulting in a defect of sporulation, and
wherein the one or more genes resulting in a defect in sporulation;
is selected from the group consisting of: kinA, kinB, spo0A, spo0B, spo0E, spo0F, spo0J, spo0M spoIIB, spoIID, spoIIE, spoIIF, spoIIG, spoIIL, spoIIM, spoIIIA, spoIIIB, spoIIIE, spoIVA, spoIVC, spoIVD, spoVG, spoVK, spoVL, spoVM, spoVN, spoVP, spoVQ, spoVID, σH, σF, σE, σG, and σK; or
is spo0A.

9-11. (canceled)

12. The composition of claim 1, wherein the composition:
further comprises a pharmaceutical carrier or excipient;
is encapsulated by a pharmaceutical grade capsule in a dry powdered form; or
is orally-available.

13-14. (canceled)

15. A method for producing an anthelmintic composition, the method comprising:
exposing a non-sporulating bacterium to an antimicrobial agent, thereby killing or inactivating the bacterium, and
optionally formulating the killed or inactivated bacterium in an orally-available dosage form,
wherein the bacterium is genetically engineered to express Cry14Ab, and
wherein the bacterium has a genetic mutation such that Cry14Ab is trapped in the cytosol of the bacterium,
optionally wherein the genetic mutation results in a defect of sporulation.

16. (canceled)

17. The method of claim 15 wherein the formulating comprises one or both steps of: of lyophilizing or spray drying the bacterium; and encapsulating the bacterium in a pharmaceutical-grade capsule.

18. (canceled)

19. The method of claim 15, wherein the antimicrobial agent is selected from one or both of an antimicrobial compound and gamma irradiation.

20. The method of claim 15, wherein the antimicrobial agent is:
(a) a food-grade antibiotic;
(b) a beta-lactam antibiotic; or
(c) a terpene, iodine or formaldehyde.

21. The method of claim 20, wherein the terpene is:
selected from the group consisting of thymol, eugenol, geraniol, carvacrol, and citral, and combinations thereof; or
wherein the terpene is carvacrol.

22. (canceled)

23. The method of claim 15, wherein CryAb1 expression is under control of a non-sporulation specific promoter, wherein the non-sporulation specific promoter is optionally a Cry3A, GerA, GNAT, or a TadA promoter.

24. (canceled)

25. The method of claim 15, wherein the inactivated bacterium is:
(a) Bacillus sp;
(b) Bacillus thuringiensis (Bt);
(c) a Gram-negative bacterium; or
(d) an E. Coli or P. Fluorescens species.

26. (canceled)

27. The method of claim 15, wherein the genetic mutation is a deletion or inactivation of one or more genes resulting in a defect of sporulation;
wherein the one or more genes;
is selected from the group consisting of: kinA, kinB, spo0A, spo0B, spo0E, spo0F, spo0J, spo0M spoIIB, spoIID, spoIIE, spoIIF, spoIIG, spoIIL, spoJIM, spoIIIA, spoIIIB, spoIIIE, spoIVA, spoIVC, spoJVD, spoVG, spoVK, spoVL, spoVM, spoVN, spoVP, spoVQ, spoVID, σH, σF, σE, σG, and σK or is spo0A.

28-30. (canceled)

31. A method of treating a parasitic worm infection in a domesticated animal or a human comprising
administering an effective amount of a composition comprising a killed or inactivated non-sporulating bacterium that is genetically engineered to express Cry14Ab in the cytosol of the bacterium to the domesticated animal or the human, wherein the administration is optionally oral.

32. The method of claim 31, wherein the parasitic worm infecting the domestic animal or the human is resistant to one or more other anthelmintic treatment, optionally wherein the parasitic work is resistant to Cry5B.

33. (canceled)

34. The method of claim 31, wherein:
the parasitic worm infecting the human is selected from roundworm, whipworm, hookworm, flatworm, tapeworm, flukes, and pinworm (threadworm);
the parasitic worm infecting the domestic animal is selected from the group consisting of roundworm, hookworm, whipworm, heartworm, lungworm, and a strongyle (cyathostomin);
the parasitic worm is a roundworm;
the domestic animals is selected from the group consisting of cattle, sheep, goats, equines, pigs, poultry, dogs and cats;
the domestic animal is a sheep and the parasitic worm is Haemonchus contortus;
the domestic animals is an equine and the parasitic worm is a strongyle (cyathostomin);
the domestic animal is a pig and the parasitic worm is a roundworm, wherein the roundworm is optionally Ascaris spp or Ascaris suum;
the parasitic worm infecting the human is selected from the group consisting of Ancylostoma spp, Necator spp, Ascaris spp, and Thrichuris spy; or
the parasitic worm infecting the human is selected from the group consisting of Ancylostoma ceylanicum, Ancylostoma duodenale, Necator americanus, Ascaris lumbricoides, and Thrichuris trichiura.

35-50. (canceled)

51. A method of controlling or preventing parasitic worm infections in domestic animals comprising feeding the domestic animals an animal feed composition comprising a base animal feed and a composition comprising a killed or inactivated non-sporulating bacterium that is genetically engineered to express Cry14Ab in the cytosol of the bacterium;
(B) a method of promoting gut health or boosting immunity in domestic animals comprising feeding the domestic animals an animal feed composition comprising a base animal feed and a composition comprising a killed or inactivated non-sporulating bacterium that is genetically engineered to express Cry14Ab in the cytosol of the bacterium, wherein the feed composition is optionally preserved in storage;
(C) a method of waste treatment, comprising contacting waste with a composition comprising a killed or inactivated non-sporulating bacterium that is genetically engineered to express Cry14Ab in the cytosol of the bacterium, wherein the waste is optionally in animal litter or in animal bedding; or
(D) A method of treating a parasitic worm infection in a domesticated animal or human, comprising orally administering an effective amount of the composition comprising a killed or inactivated non-sporulating bacterium that is genetically engineered to express Cry14Ab in the cytosol of the bacterium to a domestic animal or a human infected with a parasitic worm.

52-56. (canceled)

57. The composition of claim 1, wherein the one or more additional nematicidal proteins is Cry5B.

58-65. (canceled)
